SEQUENCE LISTING (26 sequences)

SEQ ID NO: 1 - DNA, 21 bp, artificial
  oligonucleotide encoding a sh Vps39 clone
ccacactctctggtgctgaac

SEQ ID NO: 2 - DNA, 21 bp, artificial
  oligonucleotide encoding a shSlc7a5 clone
ggcattggcttcgccatcatc

SEQ ID NO: 3 - DNA, 21 bp, artificial
  oligonucleotide encoding a shSlc7a5 clone
cgcaatatcacgctgctcaac

SEQ ID NO: 4 - DNA, 21 bp, artificial
  oligonucleotide encoding a shSlc7a5 clone
agcagaagttgtcctttgaag

SEQ ID NO: 5 - protein, 12 aa, artificial
  lymphocytic choriomeningitis virus (LCMV) polypeptide
DIYKGVYQFKSV

SEQ ID NO: 6 - protein, 9 aa, artificial
  LCMV polypeptide
KAVYNFATC

SEQ ID NO: 7 - DNA, 19 bp, artificial
  oligonucleotide primer
ccgtgaactgctacagcgt

SEQ ID NO: 8 - DNA, 20 bp, artificial
  oligonucleotide primer
cttcccgatctggacgaagc

SEQ ID NO: 9 - DNA, 20 bp, artificial
  oligonucleotide primer
gtcctgctcaacattgggca

SEQ ID NO: 10 - DNA, 19 bp, artificial
  oligonucleotide primer
cagggcctgcattctcacg

SEQ ID NO: 11 - DNA, 22 bp, artificial
  oligonucleotide primer
ccaggatggttcttagactccc

SEQ ID NO: 12 - DNA, 21 bp, artificial
  oligonucleotide primer
tttagcacgaagctctccgat

SEQ ID NO: 13 - DNA, 20 bp, artificial
  oligonucleotide primer
ccagggagcaagacagagac

SEQ ID NO: 14 - DNA, 20 bp, artificial
  oligonucleotide primer
gagactggcgttctccaaag

SEQ ID NO: 15 - DNA, 20 bp, artificial
  oligonucleotide primer
gacacaggcacttccctcat

SEQ ID NO: 16 - DNA, 20 bp, artificial
  oligonucleotide primer
ctctggggacagcttgtagc

SEQ ID NO: 17 - DNA, 20 bp, artificial
  oligonucleotide primer
acggaggagtaccaccacag

SEQ ID NO: 18 - DNA, 20 bp, artificial
  oligonucleotide primer
gcaattctgaggctctgacc

SEQ ID NO: 19 - DNA, 20 bp, artificial
  oligonucleotide primer
tctctcagtgcccagaacct

SEQ ID NO: 20 - DNA, 20 bp, artificial
  oligonucleotide primer
gccacagcttctttcaggac

SEQ ID NO: 21 - DNA, 22 bp, artificial
  oligonucleotide primer
gacccagaagcgagagctcaca

SEQ ID NO: 22 - DNA, 22 bp, artificial
  oligonucleotide primer
tgtgattgtctttcttctgccg

SEQ ID NO: 23 - DNA, 21 bp, artificial
  oligonucleotide primer
cgccgctagaggtgaaattct

SEQ ID NO: 24 - DNA, 21 bp, artificial
  oligonucleotide primer
cgaacctccgactttcgttct

SEQ ID NO: 25 - DNA, 21 bp, artificial
  oligonucleotide encoding a Tfeb shRNA
cggcagtactatgactatgat

SEQ ID NO: 26 - DNA, 21 bp, artificial
  oligonucleotide encoding a Vps39 shRNA
agtgagcatgtgctgaagaag

(19) United States
(12) Patent Application Publication
(10) Pub. No.: US 2024/0197874 A1
(43) Pub. Date: Jun. 20, 2024

(54) MATERIALS AND METHODS FOR IMPROVING EFFICACY OF ADOPTIVE IMMUNE CELL THERAPY

(71) Applicant: Mayo Foundation for Medical Education and Research, Rochester, MN (US)

(72) Inventors: Joerg J. Goronzy, Palo Alto, CA (US); Cornelia M. Weyand, Rochester, MN (US); Jun Jin, Rochester, MN (US)

(21) Appl. No.: 18/287,909

(22) PCT Filed: Jun. 1, 2022
(86) PCT No.: PCT/US2022/031753

§ 371 Date: Oct. 23, 2023

Related U.S. Application Data
(60) Provisional application No. 63/210,709, filed on Jun. 15, 2021.
(60) Provisional application No. 63/197,188, filed on Jun. 4, 2021.

(51) Int. Cl. A61K 39/00; A61K 35/17; A61P 35/00; C12N 5/0783

(52) U.S. Cl. CPC A61K 39/4631; A61K 35/17; A61K 39/4611; A61P 35/00; C12N 5/0636; A61K 2239/57; C12N 2510/00

(57) ABSTRACT

Methods and materials for treating cancer (e.g., melanoma) in a subject and for improving efficacy of adoptive immune cell therapy are described. The methods can include administering immune cells (e.g., chimeric antigen receptor T cells or tumor-infiltrating lymphocytes) having reduced expression of a VPS39 polypeptide to the subject.

### CROSS-REFERENCE TO RELATED APPLICATIONS

This application claims the benefit of U.S. Patent Application Ser. No. 63/197,188, filed on Jun. 4, 2021, and of U.S. Patent Application Ser. No. 63/210,709, filed on Jun. 15, 2021. The disclosures of the prior applications are considered part of (and are incorporated by reference in) the disclosure of this application.

### STATEMENT OF FEDERALLY SPONSORED RESEARCH

This invention was made with government support under Grant No. AG045779, awarded by National Institute of Aging. The government has certain rights in the invention.

### SEQUENCE LISTING

This document includes a Sequence Listing that has been submitted electronically as an ASCII text file named 07039-2055WO1_ST25.txt. The ASCII text file, created on May 19, 2022, is 6 kilobytes in size. The material in the ASCII text file is hereby incorporated by reference in its entirety.

### BACKGROUND

### 1. Technical Field

This document relates to methods and materials for treating cancer in a subject and improving efficacy of adoptive immune cell therapy by engineering the immune cells (e.g., chimeric antigen receptor T (CAR-T) cells or tumor-infiltrating lymphocytes) to have reduced expression of a VPS39 polypeptide and administering the engineered immune cells to a subject.

### 2. Background Information

Adoptive transfer of immune cells is a personalized form of cancer therapy. In adoptive transfer, T cells can be harvested from a patient, grown in vitro and selected, for example, for recognition of the tumor. The T cells also can be manipulated before cell transfer to enhance antitumor immunity. See, e.g., Rosenberg and Restifo, Science, 348(6230): 62-68 (2015).

### SUMMARY

This document is based, at least in part, on reducing expression of Vps39 in immune cells (e.g., chimeric antigen receptor T cells or tumor-infiltrating lymphocytes). Targeting VPS39 can be useful in clinical settings where adoptive T cell therapy is applied, such as treatment with CAR-T cells or tumor-infiltrating T cells.

In some embodiments, a method for treating cancer (e.g., melanoma) in a subject is provided. The method can include administering, to the subject, engineered immune cells, wherein the immune cells have reduced expression of a VPS39 polypeptide. The engineered immune cells can be chimeric antigen receptor T cells or tumor infiltrating lymphocytes.

This document also features a method of increasing efficacy of adoptive cell transfer in a subject. The method includes administering to the subject (e.g., a subject having cancer), chimeric antigen receptor T cells having reduced expression of a VPS39 polypeptide.

In another aspect, this document features an immune cell comprising an inactivated VPS39 gene. The immune cell can be a T cell, a chimeric antigen receptor T cell, or a tumor infiltrating lymphocyte.

This document also features an isolated immune cell that includes a disrupted nucleic acid encoding a VPS39 polypeptide, wherein the cell does not express an endogenous VPS39 polypeptide. The immune cell can be a T cell, a chimeric antigen receptor T cell, or a tumor infiltrating lymphocyte.

Unless otherwise defined, all technical and scientific terms used herein have the same meaning as commonly understood by one of ordinary skill in the art to which this disclosure pertains. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A-1K. Lysosome-independent activation of mTORC1 in naïve CD4 T cell responses. (FIG. 1A) Naïve CD4 T cells were activated with anti-CD3/anti-CD28 beads for 5 days followed by DQ-BSA treatment for 6 hours. Fluorescence of cleaved DQ-BSA was analyzed by flow cytometry to determine lysosomal activities (left). Phospho-S6K1 (Thr389) protein expression was determined by Western blotting (right). Results are from ten young (Y, 20-35 years) and ten old (O, 65-85 years) healthy individuals. Intensities of p-S6K1 protein expression were normalized to ß-actin and are shown relative to the mean from young individuals; comparison by two-tailed unpaired t-test. (FIG. 1B) Naïve CD4 T cells from young individuals were activated for 5 days with the last 2 days in the presence of vehicle, chloroquine (CQ) or bafilomycin A1 (BafA1). Lysosomal activities (left) and mTORC1 activities (right) were determined as in (FIG. 1A). (FIG. 1C) Naïve CD4 T cells from young individuals were transfected with control or TFEB siRNA and activated for 5 days. Lysosomal activities (left) and mTORC1 activities (right) were determined. (FIG. 1D) Heat map showing expression differences of genes involved in the amino acid signaling arm of the mTORC1 pathway, comparing the transcriptome of day 5-activated naïve CD4 T cells from old and young adults (Accession number: SRA: SRP158502). (FIG. 1E) SLC7A5 and SLC7A1 transcripts from day 5-activated naïve CD4 T cells from twelve 20-35 year-old and twelve 65-85 year-old healthy adults. Results are expressed relative to the mean from young individuals; comparison by two-tailed unpaired t-test. (FIG. 1F) Chromatin accessibility at SLC745 gene in human Epstein-Barr (EBV), varicella-zoster (VZV) and influenza (Flu) virus-specific CD4 T cells from young and old adults. Averaged tracks (young n=3, old n=4) at SLC745 show increased peak in highlighted (red) region. (FIG. 1G) SLC7A5 and SLC7A1 transcripts in samples from (FIG. 1B) and (FIG. 1C). (FIG. 1H) SLC7A5 and c-MYC protein expression in samples from (FIG. 1B) and (FIG. 1C). (FIG. 1I) c-MYC protein expression in samples from (FIG. 1A). (FIG. 1J) GSEA comparing fold transcript differences in young compared with old naïve CD4 T cells on day 5 after stimulation (Accession number: SRA: SRP158502) with that of “HALLMARK_MYC_TARGETS”. (FIG. 1K) SLC7A5 and c-MYC protein expression in day 5-stimulated naïve CD4 T cells from three old individuals transfected with control or MYC siRNA. *p<0.05, **p<0.01, ***p<0.001, ****p<0.0001; NS, not significant.

FIGS. 2A-2H. Naïve CD4 T cells from old adults lose lysosomal function but maintain mTORC1 activity after activation on day 5. (FIG. 2A and FIG. 2B) Naïve CD4 T cells from young and old individuals were activated with anti-CD3/anti-CD28 beads for 5 days followed by DQBSA treatments for 6 hours. Fluorescence of cleaved DQ-BSA was analyzed by flow cytometry to determine lysosome activities (FIG. 2A). Phospho-S6K1 (Thr389) protein expression (right) was determined by Western blotting (FIG. 2B). (FIG. 2C) Naïve CD4 T cells were activated for 5 days in the presence of increasing doses of chloroquine (CQ), bafilomycin A1 (BafA1) or JPH203. The percentage of live cells was determined as the frequency of Annexin V negative population. Mean+SD of four experiments. (FIGS. 2D-2G) Naïve CD4 T cells were activated for 5 days with the last 2-day culture with vehicle, chloroquine (CQ) or bafilomycin A1 (BafA1). Alternatively, cells were transfected with control or TFEB siRNA and activated for 5 days. (FIG. 2D) Lysosomal activities were determined as in (FIG. 2A). (FIG. 2E) mTORC1 activity was determined as in (FIG. 2B). (FIG. 2F) TFEB protein silencing were confirmed by Western blotting on day 5 after activation. (FIG. 2G) AKT phosphorylation was determined by Western blotting on day 5 after activation. **p<0.01; NS, not significant. (FIG. 2H) Naïve CD4 T cells from three old individuals were activated and treated with CQ as in (FIG. 2D). SLC7A5 and c-MYC protein expression were determined. Comparison by two-tailed paired t test. *p<0.05, **p<0.01; NS, not significant.

FIGS. 3A-3H. SLC7A5-dependent mTORC1 activation in naïve CD4 T cell responses from old adults. (FIGS. 3A and 3B) Longitudinal analysis of SLC7A5 gene (FIG. 3A) and protein (FIG. 3B) expression in naïve CD4 T cells from four young and four old individuals after stimulation. (FIG. 3C) SLC7A5 protein expression in ex vivo-sorted naïve CD4 T cells and central memory (CM) CD4 T cells from four young and four old individuals. Statistical significance by two-tailed unpaired t test. (FIG. 3D) Dose dependent analysis of mTORC1 activities after JPH203 inhibition as shown by the phosphorylation of S6RP. Mean+SD of four experiments. (FIGS. 3E and 3F) Naïve CD4 T cells from young and old individuals were transfected with control or SLC7A5 siRNA and activated with anti-CD3/anti-CD28 beads for 3 or 5 days. Reduced SLC7A5 protein expression after silencing was confirmed on day 3-stimulated cells from young and old individuals (FIG. 3E). mTORC1 activities were determined by flow cytometry-based analysis of intracellular p-S6RP (S235/S236) (FIG. 3F). Comparison by two-tailed paired t test. (FIGS. 3G and 3H) Naïve CD4 T cells from young individuals were transfected with control vector (pCMV6) or SLC7A5 vector (pSLC7A5, both Origene) and activated with anti-CD3/anti-CD28 beads for 5 days. SLC7A5 protein overexpression was confirmed on day 5 (FIG. 3G). mTORC1 activities were determined by flow cytometry-based analysis of intracellular p-S6RP (S235/S236) (FIG. 3H). Comparison by two-tailed paired t test. *p<0.05, **p<0.01; NS, not significant.

FIGS. 4A-4G. SLC7A5-dependent late endosomal mTORC1 activation in naïve CD4 T cell responses. (FIGS. 4A and 4B) Naïve CD4 T cells from young and old healthy individual were activated with anti-CD3/anti-CD28 beads for 3 days in the presence of vehicle or SLC7A5 inhibitor JPH203 (upper panel). Alternatively, cells were activated with anti-CD3/anti-CD28 beads for 5 days with the last 2 days in the presence of vehicle or JPH203 (lower panel). mTORC1 activities were determined either by Western blotting of p-S6K1 (FIG. 4A) or by flow cytometry of intracellular p-S6RP (S235/S236) (FIG. 4B). Data are shown as one representative experiment (left) and cumulative data of three or four experiments (right). (FIG. 4C) Naïve CD4 T cells were activated for 3 days followed by treatment or not with an AKT inhibitor for 2 hours prior to harvesting. Endosomes were isolated and analyzed for in vitro mTORC1 kinase activity toward S6K1. Total cell lysates (T) and endosome isolates (E) were analyzed by immunoblotting for indicated proteins. Data are shown as one representative of three experiments. (FIG. 4D) In vitro mTORC1 kinase activity of endosome isolates in day 5-stimulated naïve CD4 T cells from one young and one old individual. Data are shown as one representative of three experiments. (FIG. 4E) Cells were stained with anti-EEA1, anti-LAMP1 and anti-mTOR. Confocal images representative of two independent experiments are shown. Scale bar, 5 μm. (FIGS. 4F and 4G) mTORC1 activities in day 3- and day 5-stimulated naïve CD4 T cells from young and old individuals after control or VPS39 silencing. Two-tailed paired t-test. The gray histogram represents isotype control. *p<0.05, **p<0.01; NS, not significant.

FIGS. 5A-5C. VPS39 silencing does not affect TGF-β/SMAD signaling in naïve CD4 T cell responses. Naïve CD4 T cells were transfected with control or VPS39 siRNA and activated for 3 days in the presence or absence of 10 ng/ml of TGF-ß1. (FIG. 5A) VPS39 protein expression after silencing. (FIG. 5B) SMAD2 (pS465/pS467)/SMAD3 (sS423/pS425) were determined by flow-cytometry. Date are shown as frequencies of p-SMAD2/3″ cells and MFI of p-SMAD2/3″. (FIG. 5C) TGF-β/SMAD signaling activities were determined by an SMAD binding element (SBE) reporter assay. The normalized luciferase activity was obtained by subtracting background luminescence from the negative control followed by calculating the ratio of firefly luminescence from the SBE reporter to Renilla luminescence from the control Renilla luciferase vector. Mean±SEM of three experiments. Comparison by two-tailed paired t test. NS, not significant.

FIGS. 6A-6F. Sustained activation of late endosomal mTORC1 suppresses lysosomal activities in naïve CD4 T cell responses. (FIGS. 6A-6F) Naïve CD4 T cells from young (Y) and old (O) individuals were activated for 5 days. Indicated inhibitor or vehicle control was added for the last 2 days of culture (FIGS. 6A-6B and FIGS. 6E-6F). Alternatively, cells were transfected with control or VPS39 siRNA and then activated with anti-CD3/anti-CD28 beads for 5 days (FIGS. 6C-6D). (FIG. 6A, FIG. 6C, FIG. 6E, and FIG. 6F) Lysosomal cathepsins expressions were determined by qRT-PCR (left); results are normalized to control samples using 18S rRNA as internal control; mean+SD of four experiments; comparison by two-tailed paired t-test. Lysosomal activities were determined by flow cytometry-based analysis of cells treated with 5 μg/mL of DQ-BSA for 6 hours. Results are shown as representative histograms (middle) and cumulative data from four experiments (right, two-tailed paired t-test). The gray histogram represents DQ-BSA free samples. (FIGS. 6B and 6D) Cells were treated with DQ-BSA (green) and stained with anti-pS6RP (red). Confocal images representative of two independent experiments show an inverse relationship between mTORC1 and lysosomal activity. Scale bar, 20 μm. *p<0.05, **p<0.01; NS, not significant.

FIGS. 7A-7C. Effects of mTORC1 inhibition on TFEB expression and phosphorylation. (FIGS. 7A and 7B) Naïve CD4 T cells from three old individuals were activated for 3 days in the presence of vehicle or indicated inhibitor. (FIG. 7C) Alternatively, cells were transfected with control or VPS39 siRNA and activated for 3 days. pTFEB (S211), pAKT (S473), total FOX01 protein and total TFEB protein were determined byimmune blotting. TFEB transcripts were determined by quantitative RT-PCR. Comparison by two-tailed paired t test. *p<0.05, **p<0.01.

FIGS. 8A-8J. Sustained activation of late endosomal mTORC1 prevents PD-1 from lysosomal degradation. (FIGS. 8A-8D) Naïve CD4 T cells from young and old individuals were activated with anti-CD3/anti-CD28 beads for 5 days with the last 2 days in the presence of vehicle or indicated inhibitor (FIGS. 8B and 8C). Alternatively, cells were transfected with control or silencing RNA and activated for 5 days (FIGS. 8A and 8D). Representative histograms showing cell surface protein expression of PD-1 (left) and cumulative data of cell surface protein expression (middle) and gene expression (right) of PD-1; comparison by two-tailed paired t-test. (FIGS. 8E-8G) Cell surface expression (FIG. 8E), intracellular expression (FIG. 8F) and cell surface/intracellular PD-1 expression ratio (FIG. 8G) after control or VPS39 silencing in day 3-stimulated naïve CD4 T cells from old individuals. (FIG. 8H) Control or VPS39-silenced, day 5-stimulated naïve CD4 T cells from old individuals were treated with 5 μg/ml cycloheximide (CHX) to inhibit de novo PD-1 synthesis. Total PD-1 protein normalized to β-actin expression are shown as relative to non-treatment. Mean±SEM of three experiments. (FIG. 8I) Longitudinal analysis of cell surface protein expression of PD-1 in naïve CD4 T cells from ten young and ten old individuals. Mean±SEM were compared by two-tailed unpaired t-test. (FIG. 8J) PD-1 gene expression comparison between day 5-stimulated young and old naïve CD4 T cells. Two-tailed unpaired t-test. The gray histogram represents isotype control. *p<0.05, **p<0.01, ***p<0.001; NS, not significant.

FIGS. 9A-9E. Sustained activation of late endosomal mTORC1 impairs expansion of naïve CD4 T cells from old adults. (FIG. 9A) Naïve CD4 T cells from older individuals labeled with CellTrace Violet (CTV) were transfected with control or VPS39 siRNA and stimulated with anti-CD3/anti-CD28 beads for 5 days in the presence or absence of PD-L1-Fc and anti-human IgG Fc antibody to crosslink PD-1. Representative histograms (left), summary data of proliferation indices (middle) and cell numbers per culture (right); comparison by two-tailed paired t-test. (FIG. 9B) CTV-labeled naïve CD4 T cells from eight young and eight old individuals were activated by anti-CD3/anti-CD28 beads for 6 days with the last 3 day in the presence or absence of PD-1 crosslinking. Representative histograms (left), summary data of proliferation indices (middle) and cell numbers per culture (right); comparison by two-tailed unpaired t-test. The gray histogram represents unstimulated cells. (FIG. 9C) CTV-labeled PBMCs from SARS-CoV-2 unexposed healthy individuals were cultured with SARS-CoV-2 peptide megapools for CD4 and CD8 cells for 8 days. VPS39 silencing FANA ASO or scramble control were added to the culture on day 0. Representative flow plots of the frequencies of CTV low CD4 and CD8 T cells for indicated conditions and summary data from six individuals; two-tailed paired t test. (FIGS. 9D and 9E) Pertussis peptide megapool responses of PBMC from six healthy individuals under the conditions of VPS39 genetic silencing (FIG. 9D) and PD-1 blockade (FIG. 9E). Two-tailed paired t test. *p<0.05, **p<0.01, ***p<0.001; NS, not significant.

FIGS. 10A-10O. Inhibition of late endosomal mTORC1 promotes primary CD4 T cell responses after LCMV infection in vivo. (FIGS. 10A-10C) 1×10⁴ Tfeb shRNA or control shRNA retrovirally transduced Amcyan⁺ LCMV-specific naive SMARTA TCR transgenic CD4 T cells were adoptively transferred into CD45.2⁺ naive recipients followed by infection with LCMV Armstrong. On day 8 post infection, spleens were harvested and analyzed. FACS plots are gated on CD4⁺ Amcyan⁺ SMARTA cells. (FIG. 10A) Tfeb protein expression in transduced cells before adoptive transfer. (FIG. 10B) PD-1 expression on day 8 p.i. (FIG. 10C) SMARTA CD4 T cell numbers per spleen on day 8 p.i. (FIGS. 10D-10K) Analysis of T cells responses as described in (FIGS. 10A-10C) after adoptive transfer of Vps39 shRNA transduced SMARTA CD4 T cells. (FIG. 10D) Vps39 protein expression in transduced cells before adoptive transfer. (FIG. 10E) Phosphorylation of S6RP on day 8 p.i. (FIG. 10F) PD-1 expression on day 8 p.i. (FIG. 10G) SMARTA CD4 T cell numbers per spleen on day 8 p.i. (FIG. 10H) Cell apoptosis and proliferation of transferred SMARTA CD4 T cells on day 8 p.i. (FIG. 10I) Mice infected with LCMV after adoptive transfer of transduced cells were additionally treated with anti-PD-1 antibody (29F.1A12) or control IgG on days 0, 3 and 6 post infection. SMARTA CD4 T cell numbers were determined on day 8 after LCMV infection. (FIG. 10J) SMARTA CD4 T cell numbers in the spleen at day 30 after infection. (FIG. 10K) Longitudinal analysis of SMARTA cells in the spleen of LCMV-infected B6 mice. (FIGS. 10L-10O) 1×10⁴ Slc7a5 shRNA or control shRNA retrovirally transduced Amcyan⁺ LCMV-specific naive SMARTA CD4 T cells were adoptively transferred into CD45.2⁺ naive recipients as in (FIGS. 10A-10C). Phosphorylation of S6RP (FIG. 10L), PD-1 expression (FIG. 10M), SMARTA cell numbers per spleen (FIG. 10N), and cell apoptosis and proliferation (FIG. 10O) of transduced SMARTA CD4 T cells on day 8 post LCMV infection. Data are pooled from two independent experiments with 3-4 mice per group (FIGS. 10B-10C and FIGS. 10E-10G), representative of two independent experiments with 3-5 mice per group (FIGS. 10A, 10D, and 10H-10K) or one experiment with 5 mice per group (FIGS. 10L-10O). Statistical significance by two-tailed unpaired t test (FIGS. 10B-10C, 10E-10H, and 10J-10O) or one-way analysis of variance (ANOVA) followed by Tukey's multiple comparisons test (FIG. 10I). The gray histograms represent naïve cells. *p<0.05, **p<0.01, ***p<0.001, ****p<0.0001; NS, not significant.

FIGS. 11A-11G. Inhibition of late endosomal mTORC1 promotes virus-specific CD4 T cell expansion without affecting their cytokine production. (FIGS. 11A-11C) Naïve SMARTA transgenic CD4 T cells were activated in plates coated with 8 μg/ml anti-CD3 Ab and 8 μg/ml anti-CD28 Ab. Cells were transduced with a retroviral vector expressing either scrambled control shRNA, Vps39 or Slc7a5 shRNA on days 1 and 2 after activation. At day 6 after activation, retrovirus-transduced Amcyan positive SMARTA cells were purified and effects of silencing on protein expression were confirmed by Western blotting. 1×10 Vps39 shRNA or control shRNA retrovirally transduced Amcyan⁺ LCMV-specific naïve SMARTA CD4 T cells were adoptively transferred into CD45.2⁺ naïve recipients. Mice were infected with 2×10 PFU of LCMV Armstrong. On day 8 post infection, spleens were harvested and analyzed. (FIG. 11A) Lysosomal activities of SMARTA cells were determined by flow cytometry-based analysis of splenocytes treated with 5 μg/ml of DQ-BSA for 6 hours. (FIG. 11B) Cytokine production of SMARTA cells after LMCV gp66 peptide restimulation of splenocytes ex vivo. (FIG. 11C) CTLA-4 expression by SMARTA cells on day 8 after LCMV infection. (FIG. 11D) Vps39 silencing by a second sh Vps39 clone (#2, 5′-CCACACTCTCTGGTGCTGAAC-3′ (SEQ ID NO:1)). (FIG. 11E) Numbers of SMARTA cells after shVps39 #2 silencing on day 30 after LCMV infection. (FIG. 11F) Numbers of D^{b} LCMV GP33-41 (GP33) tetramer+ cells gated on endogenous CD8 T cells in the spleen of host mice at day 30 after infection. (FIG. 11G) Slc7a5 silencing by three different shSlc7a5 clones. Clone 1: 5′-GGCATTGGCTTCGCCATCATC-3′ (SEQ ID NO:2); Clone #2: 5′-: CGCAATATCACGCTGCTCAAC-3′ (SEQ ID NO:3); Clone #3: 5′ AGCAGAAGTTGTCCTTTGAAG-3′ (SEQ ID NO:4); based on its partial inhibition resembling the age-associated difference in humans, Clone #2 was chosen for subsequent studies. Data are representative of two independent experiments with 3-4 mice per group (FIGS. 11A-11C) or one experiment (FIGS. 11D-11G). Comparison by two-tailed unpaired t test. *p<0.05, **p<0.01; NS, not significant.

FIGS. 12A-12H. Inhibition of late endosomal mTORC1 augments CD4 T cell helper responses in vivo. 1×10⁴ Vps39 shRNA or control shRNA retrovirally transduced Amcyan⁺ naive SMARTA CD4 T cells were adoptively transferred into CD45.2⁺ naive recipient followed by LCMV infection. On days 8 (FIGS. 12A-12C) and 30 (FIG. 12E) post infection, spleens were harvested and analyzed. Alternatively, on day 30, immune mice were rechallenged with Lm-gp33 for 6 days (FIGS. 12F-12H). (FIG. 12A) Number of germinal center (GC) SMARTA TFH cells on day 8 after infection. (FIGS. 12B and 12C) Number of endogenous Fas⁺ GL-7⁺ GC B cells (FIG. 12B) and CD138⁺ IgD plasma cells (FIG. 12C) in host mice on day 8 after infection. Representative contour plots gated on B220⁺ CD4⁻ B cells (left) and summary data (right). (FIG. 12D) Anti-LCMV nucleoprotein IgG titers in serum on day 14 after infection. n=5 mice per group. (FIG. 12E) Numbers of D^{b} LCMV GP33-41 (GP33) tetramer⁺ cells gated on endogenous CD8 T cells in the spleen of host mice on day 30 after infection. (FIG. 12F) Numbers of endogenous D^{b} LCMV GP33 tetramer⁺ CD8 T cells in the spleen on day 6 after Lm-GP33 challenge. (FIG. 12G) Fold expansion of D^{b} GP33 tetramer+ memory CD8 T cells upon Lm-GP33 on day 6. (FIG. 12H) Cytokine production by CD8 T cells harvested on day 6 after infection and restimulated with GP33 peptide in vitro. Data are representative of two independent experiments with 4-5 mice per group (FIGS. 12A-12E) or one experiment with 5 mice per group (FIGS. 12F-12H). Statistical significance by two-tailed unpaired t test. *p<0.05, **p<0.01, ***p<0.001, ****p<0.0001; NS, not significant.

FIGS. 13A-13C. Improved CD4 T cell helper responses after Vps39 silencing depends on reduced PD-1 expression. 1×10⁴ Vps39 shRNA or control shRNA retrovirally transduced Amcyan⁺ LCMV-specific naïve SMARTA CD4 T cells were adoptively transferred into CD45.2⁺ naïve recipients followed by infection with LCMV Armstrong. Mice were treated with anti-PD-1 antibody (29F.1A12) or control IgG at days 0, 3, and 6 post infection. On day 8 post infection, spleens were harvested and analyzed. (FIG. 13A) Number of CXCR5^{hi}PD-1^{hi} SMARTA GC TFH cells. (FIG. 13B) Number of endogenous Fas⁺ IgD⁻ plasma cells in host mice (FIG. 13C). Data are from one experiment with 4 mice per group. One-way analysis of variance (ANOVA) followed by Tukey's multiple comparisons test. *p<0.05; NS, not significant.

FIGS. 14A-14G. (FIG. 14A) Schematic of experimental design to prevent melanoma growth by adoptively transferred Vps39-silenced CAR-T cells. (FIG. 14B) Tumor growth curves for B16-OVA tumors following transfer of naive OT-1 control or Vps39-silenced CD8⁺ T cells separately into wild-type recipients. n=8 mice. (FIG. 14C) Survival curves of mice in B. (FIG. 14D) Cell surface PD-1 expression of tumor-infiltrating Amcyan⁺ OT-I cells at day 9 after tumor implant. (FIG. 14E) Numbers of tumor-infiltrating Amcyan⁺ OT-I cells at day 9 after tumor implant. (FIG. 14F) Cytokine production of tumor-infiltrating Amcyan⁺ OT-I cells restimulated with OVA257-264 peptide ex vivo at day 9 after tumor implant. (FIG. 14G) Alternatively, at day 18 after tumor implant, Amcyan⁺ tumor-infiltrating OT-I cells were sorted and injected into a new group of wild-type C57BL/6 mice followed by B16-OVA tumor implant. Tumor growth curves for B16-OVA tumors following transfer of OT-1 control or Vps39-silenced CD8⁺ T cells that had been challenged with the same tumor cell line for 18 days separately into wild-type recipients. n=6 mice. Data are one experiment with 6 to 8 mice per group (FIGS. 14B, 14C, and 14G) or one experiment with four mice per group (FIGS. 14D-14F). Statistical significance by two-tailed unpaired t test. *P<0.05, **P<0.01, ***P<0.001, and ****P<0.0001.

### DETAILED DESCRIPTION

This document provides methods and materials for engineering immune cells (e.g., T cells such as chimeric antigen receptor (CAR) T cells (CAR-T cells) or tumor-infiltrating lymphocytes (TILs)) to have reduced expression of a VPS39 polypeptide. VPS39 is part of the complex that mediates fusion of autophagosomes with lysosomes (HOPS complex). In some embodiments, expression of VPS11, VPS16, VPS18, VPS33, or VPS41 can be reduced in an immune cell.

In some cases, a T cell (e.g., CAR T cells) can be engineered to knock out (KO) a nucleic acid encoding a VPS39 polypeptide to reduce VPS39 polypeptide expression in that T cell (e.g., as compared to a T cell that is not engineered to KO a nucleic acid encoding a VPS39 polypeptide). A T cell that is engineered to KO a nucleic acid encoding a VPS39 polypeptide can also be referred to herein as a VPS39 KO T cell. In some cases, the methods and materials provided herein can be used to treat cancer or improve the efficacy of adoptive transfer of immune cells.

The term “reduced level” as used herein with respect to an expression level of VPS39 refers to any level that is lower than a reference expression level of VPS39. The term “reference level” as used herein with respect to VPS39 refers to the level of VPS39 typically observed in a sample (e.g., a control sample) from one or more mammals (e.g., humans) not engineered to have a reduced expression level of VPS39 as described herein. Control samples can include, without limitation, T cells that are wild-type T cells (e.g., T cells that are not VPS39 KO T cells). In some cases, a reduced expression level of a VPS39 polypeptide can be an undetectable level of VPS39. In some cases, a reduced expression level of VPS39 polypeptides can be an eliminated level of VPS39.

In some cases, a T cell having (e.g., engineered to have) a reduced level of a VPS39 polypeptide (e.g., a VPS39 KO T cell) can have enhanced CAR T cell function such as antitumor activity, enhanced T cell expansion, proliferation, cell killing, cytokine production, exhaustion susceptibility, antigen specific effector functions, persistence, and differentiation (e.g., as compared to a CAR T cell that is not engineered to have a reduced level of VPS39 polypeptides as described herein).

A T cell having (e.g., engineered to have) a reduced expression level of a VPS39 polypeptide such as a VPS39 KO T cell can be any appropriate T cell. A T cell can be a naïve T cell. Examples of T cells that can be designed to have a reduced expression level of VPS39 as described herein include, without limitation, cytotoxic T cells (e.g., CD4+ CTLs and/or CD8+ CTLs). For example, a T cell that can be engineered to have a reduced level of a VPS39 polypeptide as described herein can be a CAR T cell. In some cases, one or more T cells can be obtained from a mammal (e.g., a mammal having cancer). For example, T cells can be obtained from a mammal to be treated with the materials and method described herein.

A T cell having (e.g., engineered to have) a reduced expression level of a VPS39 polypeptide can be generated using any appropriate method. In some cases, a T cell (e.g., a CAR T cell) can be engineered to KO a nucleic acid encoding a VPS39 polypeptide to reduce VPS39 polypeptide expression in that T cell. Examples of techniques that can be used to knock out a nucleic acid sequence encoding a VPS39 polypeptide include, without limitation, gene editing, homologous recombination, non-homologous end joining, and microhomology end joining. For example, gene editing (e.g., with engineered nucleases) can be used to KO a nucleic acid encoding a VPS39 polypeptide. Nucleases useful for genome editing include, without limitation, CRISPR-associated (Cas) nucleases, zinc finger nucleases (ZFNs), transcription activator-like effector (TALE) nucleases, and homing endonucleases (HE; also referred to as meganucleases).

In some cases, a clustered regularly interspaced short palindromic repeat (CRISPR)/Cas system can be used (e.g., can be introduced into one or more T cells) to KO a nucleic acid encoding a VPS39 polypeptide. A CRISPR/Cas system used to KO a nucleic acid encoding a VPS39 polypeptide can include any appropriate guide RNA (gRNA). In some cases, a gRNA can be complementary to a nucleic acid encoding a VPS39 polypeptide.

A CRISPR/Cas system used to KO a nucleic acid encoding a VPS39 polypeptide can include any appropriate Cas nuclease. Examples of Cas nucleases include, without limitation, Cas1, Cas2, Cas3, Cas9, Cas10, and Cpf1. In some cases, a Cas component of a CRISPR/Cas system designed to KO a nucleic acid encoding a VPS39 polypeptide can be a Cas9 nuclease. For example, the Cas9 nuclease of a CRISPR/Cas9 system described herein can be a lentiCRISPRv2 (see, e.g., Shalem et al., 2014 Science 343:84-87; and Sanjana et al., 2014 Nature methods 11: 783-784).

Components of a CRISPR/Cas system (e.g., a gRNA and a Cas nuclease) used to KO a nucleic acid encoding a VPS39 polypeptide can be introduced into one or more T cells (e.g., CAR T cells) in any appropriate format. In some cases, a component of a CRISPR/Cas system can be introduced into one or more T cells as a nucleic acid encoding a gRNA and/or a nucleic acid encoding a Cas nuclease. For example, a nucleic acid encoding at least one gRNA (e.g., a gRNA sequence specific to a nucleic acid encoding a VPS39 polypeptide) and a nucleic acid at least one Cas nuclease (e.g., a Cas9 nuclease) can be introduced into one or more T cells. In some cases, a component of a CRISPR/Cas system can be introduced into one or more T cells as a gRNA and/or as a Cas nuclease. For example, at least one gRNA (e.g., a gRNA sequence specific to a nucleic acid encoding a VPS39 polypeptide) and at least one Cas nuclease (e.g., a Cas9 nuclease) can be introduced into one or more T cells.

In some cases, when components of a CRISPR/Cas system (e.g., a gRNA and a Cas nuclease) are introduced into one or more T cells as nucleic acid encoding the components (e.g., nucleic acid encoding a gRNA and nucleic acid encoding a Cas nuclease), the nucleic acid can be any appropriate form. For example, a nucleic acid can be a construct (e.g., an expression construct). A nucleic acid encoding at least one gRNA and a nucleic acid encoding at least one Cas nuclease can be on separate nucleic acid constructs or on the same nucleic acid construct. In some cases, a nucleic acid encoding at least one gRNA and a nucleic acid encoding at least one Cas nuclease can be on a single nucleic acid construct. A nucleic acid construct can be any appropriate type of nucleic acid construct. Examples of nucleic acid constructs that can be used to express at least one gRNA and/or at least one Cas nuclease include, without limitation, expression plasmids and viral vectors (e.g., lentiviral vectors). In cases where a nucleic acid encoding at least one gRNA and a nucleic acid encoding at least one Cas nuclease are on separate nucleic acid constructs, the nucleic acid constructs can be the same type of construct or different types of constructs. In some cases, a nucleic acid encoding at least one gRNA sequence specific to a nucleic acid encoding a VPS39 polypeptide and a nucleic acid encoding at least one Cas nuclease can be on a single lentiviral vector and used in ex vivo engineering of T cells to have a reduced expression level of VPS39.

In some cases, components of a CRISPR/Cas system (e.g., a gRNA and a Cas nuclease) can be introduced directly into one or more T cells (e.g., as a gRNA and/or as Cas nuclease). A gRNA and a Cas nuclease can be introduced into the one or more T cells separately or together. In cases where a gRNA and a Cas nuclease are introduced into the one or more T cells together, the gRNA and the Cas nuclease can be in a complex. When a gRNA and a Cas nuclease are in a complex, the gRNA and the Cas nuclease can be covalently or non-covalently attached. In some cases, a complex including a gRNA and a Cas nuclease also can include one or more additional components. Examples of complexes that can include components of a CRISPR/Cas system (e.g., a gRNA and a Cas nuclease) include, without limitation, ribonucleoproteins (RNPs) and effector complexes (e.g., containing a CRISPR RNAs (crRNAs) a Cas nuclease). For example, at least one gRNA and at least one Cas nuclease can be included in a RNP. In some cases, a RNP can include gRNAs and Cas nucleases at a ratio of about 1:1 to about 10:1 (e.g., about 1:1 to about 10:1, about 2:1 to about 10:1, about 3:1 to about 10:1, about 5:1 to about 10:1, about 8:1 to about 10:1, about 1:1 to about 9:1, about 1:1 to about 7:1, about 1:1 to about 5:1, about 1:1 to about 4:1, about 1:1 to about 3:1, about 1:1 to about 2:1, about 2:1 to about 8:1, about 3:1 to about 6:1, about 4:1 to about 5:1, or about 5:1 to about 7:1). For example, a RNP can include gRNAs and Cas nucleases at about a 1:1 ratio. For example, a RNP can include gRNAs and Cas nucleases at about a 2:1 ratio. In some cases, a RNP including at least one gRNA sequence specific to a nucleic acid encoding a VPS39 polypeptide and at least one Cas9 nuclease can be used in ex vivo engineering of T cells to have a reduced level of a VPS39 polypeptide.

Components of a CRISPR/Cas system (e.g., a gRNA and a Cas nuclease) used to KO a nucleic acid encoding a VPS39 polypeptide can be introduced into one or more T cells (e.g., CAR T cells) using any appropriate method. A method of introducing components of a CRISPR/Cas system into a T cell can be a physical method. A method of introducing components of a CRISPR/Cas system into a T cell can be a chemical method. A method of introducing components of a CRISPR/Cas system into a T cell can be a particle-based method. Examples of methods that can be used to introduce components of a CRISPR/Cas system into one or more T cells include, without limitation, electroporation, transfection (e.g., lipofection), transduction (e.g., viral vector mediated transduction), microinjection, and nucleofection. In some cases, when components of a CRISPR/Cas system are introduced into one or more T cells as nucleic acid encoding the components, the nucleic acid encoding the components can be transduced into the one or more T cells. For example, a lentiviral vector encoding at least one gRNA sequence specific to a nucleic acid encoding a VPS39 polypeptide and at least one Cas9 nuclease can be transduced into T cells (e.g., ex vivo T cells). In some cases, when components of a CRISPR/Cas system are introduced directly into one or more T cells, the components can be electroporated into the one or more T cells. For example, a RNP including at least one gRNA sequence specific to a nucleic acid encoding a VPS39 polypeptide and at least one Cas9 nuclease can be electroporated into T cells (e.g., ex vivo T cells). In some cases, components of a CRISPR/Cas system can be introduced ex vivo into one or more T cells. For example, ex vivo engineering of T cells to have a reduced level of a VPS39 polypeptide can include transducing isolated T cells with a lentiviral vector encoding components of a CRISPR/Cas system. For example, ex vivo engineering of T cells having reduced levels of a VPS39 polypeptide can include electroporating isolated T cells with a complex including components of a CRISPR/Cas system. In cases where T cells are engineered ex vivo to have a reduced level of VPS39 polypeptide, the T cells can be obtained from any appropriate source (e.g., a mammal such as the mammal to be treated or a donor mammal, or a cell line).

In some cases, a T cell (e.g., a CAR T cell) can be treated with one or more inhibitors of VPS39 polypeptide expression to reduce VPS39 polypeptide expression in that T cell (e.g., as compared to a T cell that was not treated with one or more inhibitors of VPS39 polypeptide expression). An inhibitor of VPS39 polypeptide expression can be any appropriate inhibitor, including without limitation, nucleic acid molecules designed to induce RNA interference (e.g., a siRNA molecule or a shRNA molecule), antisense molecules, miRNAs, receptor blockade, and antibodies (e.g., antagonistic antibodies and neutralizing antibodies).

A T cell having (e.g., engineered to have) a reduced expression level of a VPS39 polypeptide can express (e.g., can be engineered to express) any appropriate antigen receptor. In some cases, an antigen receptor can be a heterologous antigen receptor. In some cases, an antigen receptor can be a CAR. In some cases, an antigen receptor can be a tumor antigen (e.g., tumor-specific antigen) receptor. For example, a T cell can be engineered to express a tumor-specific antigen receptor that targets a tumor-specific antigen (e.g., a cell surface tumor-specific antigen) expressed by a cancer cell in a mammal having cancer. Examples of antigens that can be recognized by an antigen receptor expressed in a T cell having reduced expression of a VPS39 polypeptide as described herein include, without limitation, cluster of differentiation 19 (CD19), mucin 1 (MUC-1), human epidermal growth factor receptor 2 (HER-2), estrogen receptor (ER), epidermal growth factor receptor (EGFR), alphafetoprotein (AFP), carcinoembryonic antigen (CEA), CA-125, epithelial tumor antigen (ETA), melanoma-associated antigen (MAGE), CD33, CD123, CLL-1, E-Cadherin, folate receptor alpha, folate receptor beta, IL13R, EGFRviii, CD22, CD20, kappa light chain, lambda light chain, desmopressin, CD44v, CD45, CD30, CD5, CD7, CD2, CD38, BCMA, CD138, FAP, CS-1, and C-met.

Any appropriate method can be used to express an antigen receptor on a T cell having (e.g., engineered to have) a reduced expression level of a VPS39 polypeptide. For example, a nucleic acid encoding an antigen receptor can be introduced into one or more T cells. In some cases, viral transduction can be used to introduce a nucleic acid encoding an antigen receptor into a non-dividing a cell. A nucleic acid encoding an antigen receptor can be introduced in a T cell using any appropriate method. In some cases, a nucleic acid encoding an antigen receptor can be introduced into a T cell by transduction (e.g., viral transduction using a retroviral vector such as a lentiviral vector) or transfection. In some cases, a nucleic acid encoding an antigen receptor can be introduced ex vivo into one or more T cells. For example, ex vivo engineering of T cells expressing an antigen receptor can include transducing isolated T cells with a lentiviral vector encoding an antigen receptor. In cases where T cells are engineered ex vivo to express an antigen receptor, the T cells can be obtained from any appropriate source (e.g., a mammal such as the mammal to be treated or a donor mammal, or a cell line).

In some cases, when a T cell having (e.g., engineered to have) a reduced expression level of a VPS39 polypeptide also expresses (e.g., is engineered to express) an antigen receptor, that T cell can be engineered to have a reduced expression level of VPS39 and engineered to express an antigen receptor using any appropriate method. In some cases, a T cell can be engineered to have a reduced expression level of a VPS39 polypeptide first and engineered to express an antigen receptor second, or vice versa. In some cases, a T cell can be simultaneously engineered to have a reduced expression level of a VPS39 polypeptide and to express an antigen receptor. For example, one or more nucleic acids used to reduce expression of a VPS39 polypeptide (e.g., a lentiviral vector encoding at least one gRNA sequence specific to a nucleic acid encoding VPS39 and at least one Cas9 nuclease or a nucleic acid encoding at least one oligonucleotide that is complementary to the VPS39 mRNA) and one or more nucleic acids encoding an antigen receptor (e.g., a CAR) can be simultaneously introduced into one or more T cells. One or more nucleic acids used to reduce expression of a VPS39 polypeptide and one or more nucleic acids encoding an antigen receptor can be introduced into one or more T cells on separate nucleic acid constructs or on a single nucleic acid construct. In some cases, one or more nucleic acids used to reduce expression of a VPS39 polypeptide and one or more nucleic acids encoding an antigen receptor can be introduced ex vivo into one or more T cells. In cases where T cells are engineered ex vivo to have a reduced expression levels of VPS39 and to express an antigen receptor, the T cells can be obtained from any appropriate source (e.g., a mammal such as the mammal to be treated or a donor mammal, or a cell line).

In some cases, a T cell having (e.g., engineered to have) a reduced expression level of a VPS39 polypeptide can be stimulated. A T cell can be stimulated at the same time as being engineered to have a reduced level of a VPS39 polypeptide or independently of being engineered to have a reduced level of a VPS39 polypeptide. For example, one or more T cells having a reduced level of a VPS39 polypeptide used in an adoptive cell therapy can be stimulated first, and can be engineered to have a reduced expression level of a VPS39 polypeptide second, or vice versa. In some cases, one or more T cells having a reduced expression level of a VPS39 polypeptide used in an adoptive cell therapy can be stimulated first, and can be engineered to have a reduced level of VPS39 polypeptide second. A T cell can be stimulated using any appropriate method. For example, a T cell can be stimulated by contacting the T cell with one or more CD polypeptides. Examples of CD polypeptides that can be used to stimulate a T cell include, without limitation, CD3, CD28, inducible T cell co-stimulator (ICOS), CD137, CD2, OX40, and CD27. In some cases, a T cell can be stimulated with CD3 and CD28 prior to introducing components of a CRISPR/Cas system (e.g., a gRNA and/or a Cas nuclease) to the T cell to KO a nucleic acid encoding a VPS39 polypeptide.

This document also provides methods and materials involved in treating cancer. For example, one or more T cells having (e.g., engineered to have) a reduced expression level of a VPS39 polypeptide can be administered (e.g., in an adoptive cell therapy such as a CART therapy) to a mammal (e.g., a human) having cancer to treat the mammal. In some cases, methods of treating a mammal having cancer as described herein can reduce the number of cancer cells (e.g., cancer cells expressing a tumor antigen) within a mammal. In some cases, methods of treating a mammal having cancer as described herein can reduce the size of one or more tumors (e.g., tumors expressing a tumor antigen) within a mammal.

Any appropriate mammal (e.g., a human) having a cancer can be treated as described herein. Examples of mammals that can be treated as described herein include, without limitation, humans, primates (such as monkeys), dogs, cats, horses, cows, pigs, sheep, mice, and rats. For example, a human having a cancer can be treated with one or more T cells having (e.g., engineered to have) a reduced expression level of a VPS39 polypeptide in, for example, an adoptive T cell therapy such as a CART cell therapy or TIL therapy using the methods and materials described herein.

When treating a mammal (e.g., a human) having a cancer as described herein, the cancer can be any appropriate cancer. In some cases, a cancer treated as described herein can be a solid tumor. In some cases, a cancer treated as described herein can be a hematological cancer. In some cases, a cancer treated as described herein can be a primary cancer. In some cases, a cancer treated as described herein can be a metastatic cancer. In some cases, a cancer treated as described herein can be a refractory cancer. In some cases, a cancer treated as described herein can be a relapsed cancer. In some cases, a cancer treated as described herein can express a tumor-associated antigen (e.g., an antigenic substance produced by a cancer cell). Examples of cancers that can be treated as described herein include, without limitation, B cell cancers (e.g., diffuse large B cell lymphoma (DLBCL) and B cell leukemias), acute lymphoblastic leukemia (ALL), chronic lymphocytic leukemia (CLL), follicular lymphoma, mantle cell lymphoma, non-Hodgkin lymphoma, Hodgkin lymphoma, acute myeloid leukemia (AML), multiple myeloma, head and neck cancers, sarcomas, breast cancer, gastrointestinal malignancies, bladder cancers, urothelial cancers, kidney cancers, lung cancers, prostate cancers, ovarian cancers, cervical cancers, genital cancers (e.g., male genital cancers and female genital cancers), and bone cancers.

Any appropriate method can be used to identify a mammal having cancer. For example, imaging techniques and biopsy techniques can be used to identify mammals (e.g., humans) having cancer. Once identified as having a cancer, a mammal can be administered one or more T cells having (e.g., engineered to have) a reduced expression level of a VPS39 polypeptide as described herein.

For example, one or more T cells having (e.g., engineered to have) a reduced expression level of a VPS39 polypeptide can be used in an adoptive T cell therapy (e.g., a CAR T cell therapy) to treat a mammal having a cancer. For example, one or more T cells having a reduced level of a VPS39 polypeptide can be used in an adoptive T cell therapy (e.g., a CAR T cell therapy) targeting any appropriate antigen within a mammal (e.g., a mammal having cancer). In some cases, an antigen can be a tumor-associated antigen (e.g., an antigenic substance produced by a cancer cell). Examples of tumor-associated antigens that can be targeted by an adoptive T cell therapy provided herein include, without limitation, CD19 (associated with DLBCL, ALL, and CLL), AFP (associated with germ cell tumors and/or hepatocellular carcinoma), CEA (associated with bowel cancer, lung cancer, and/or breast cancer), CA-125 (associated with ovarian cancer), MUC-1 (associated with breast cancer), ETA (associated with breast cancer), MAGE (associated with malignant melanoma), CD33 (associated with AML), CD123 (associated with AML), CLL-1 (associated with AML), E-Cadherin (associated with epithelial tumors), folate receptor alpha (associated with ovarian cancers), folate receptor feta (associated with ovarian cancers and AML), IL13R (associated with brain cancers), EGFRviii (associated with brain cancers), CD22 (associated with B cell cancers), CD20 (associated with B cell cancers), kappa light chain (associated with B cell cancers), lambda light chain (associated with B cell cancers), CD44v (associated with AML), CD45 (associated with hematological cancers), CD30 (associated with Hodgkin lymphomas and T cell lymphomas), CD5 (associated with T cell lymphomas), CD7 (associated with T cell lymphomas), CD2 (associated with T cell lymphomas), CD38 (associated with multiple myelomas and AML), BCMA (associated with multiple myelomas), CD138 (associated with multiple myelomas and AML), FAP (associated with solid tumors), CS-1 (associated with multiple myeloma), and c-Met (associated with breast cancer).

In some cases, one or more T cells having (e.g., engineered to have) a reduced expression level of a VPS39 polypeptide can be used in an adoptive T cell therapy (e.g., a CAR T cell therapy) to treat a mammal having a disease or disorder other than cancer. For example, one or more T cells having a reduced level of VPS39 polypeptide can be used in an adoptive T cell therapy (e.g., a CAR T cell therapy) targeting any appropriate disease-associated antigen (e.g., an antigenic substance produced by cell affected by a particular disease) within a mammal. Examples of disease-associated antigens that can be targeted by an adoptive T cell therapy provided herein include, without limitation desmopressin (associated with auto immune skin diseases).

In some cases, one or more T cells having (e.g., engineered to have) a reduced expression level of a VPS39 polypeptide used in an adoptive T cell therapy (e.g., a CAR T cell therapy) can be administered to a mammal having a cancer as a combination therapy with one or more additional agents used to treat a cancer. For example, one or more T cells having a reduced level of a VPS39 polypeptide used in an adoptive cell therapy can be administered to a mammal in combination with one or more anti-cancer treatments (e.g., surgery, radiation therapy, chemotherapy (e.g., alkylating agents such as busulfan), targeted therapies, hormonal therapy, angiogenesis inhibitors, and/or immunosuppressants (e.g., interleukin-6 inhibiting agents such as tocilizumab). In cases where one or more T cells having a reduced level of a VPS39 polypeptide used in an adoptive cell therapy are used with additional agents treat a cancer, the one or more additional agents can be administered at the same time or independently. In some cases, one or more T cells having a reduced level of a VPS39 polypeptide used in an adoptive cell therapy can be administered first, and the one or more additional agents administered second, or vice versa.

The invention will be further described in the following examples, which do not limit the scope of the invention described in the claims.

### EXAMPLES

### Example 1—Materials and Methods

Aging is associated with a decline in adaptive immunity, resulting in decreased efficacy of vaccination and increased morbidity from infections with newly as well as previously encountered pathogens. Most noticeable are the increased susceptibility during the annual influenza epidemic and, most recently, the infection with the SARS-Cov-2 virus. Although the increased morbidity is clearly multifactorial, age-related changes in CD4 T cell survival and differentiation have been identified that contribute to the immune decline in older individuals. CD4 T helper cells are pivotal for mounting a protective immune response after vaccination or infections. Generation of CD4 T follicular helper (TFH) cells is essential for germinal center (GC) formation, class switching and affinity maturation. Moreover, CD4 T cells are required for effective CD8 T cell memory and recall responses. Recent studies have shown a T cell-intrinsic bias towards short-lived effector cells differentiation in CD4 T cell responses of older adults at the expense of TFH and memory precursor cells. One important mechanism causing this bias is a sustained activation of the mechanistic target of rapamycin complex 1 (mTORC1).

mTORC1 plays an important role in regulating T cell responses by coordinating cell growth and cellular metabolism with environmental inputs, in particular nutrient resources, and facilitating the switch toward anabolic metabolism that is required for cell proliferation and effector cell differentiation. Complete block of mTORC1 activities by either a treatment with high dose of rapamycin or a genetic depletion of RHEB (an upstream activator of mTORC1) dramatically reduced the number of antigen-specific effector and memory precursor T cells at peak responses after primary infection RHEB-deficient memory CD8 T cells even failed to respond to secondary immunization. However, overactivation of mTORC1 by genetic depletion of TSC1 (an upstream inhibitor of mTORC1) reduced the numbers of memory precursor CD8 T cells at peak responses after primary infection instead of promoting them. Indeed, moderate inhibition of mTORC1 by pharmacological compounds produced increased clonal expansion of antigen-specific T cells after primary responses and enhanced memory recall responses after antigen rechallenge in several infection models. These data led to the conclusion that mTORC1 inhibition promotes long-term memory over short-lived effector CD8 T cell differentiation. Taken together, fine-tuning of mTORC1 activity is important for generating protective primary and recall T cell responses, in particular for older adults, who have sustained mTORC1 activity after T cell activation resulting in the preferential generation of short-lived effector T cells.

mTORC1 translocates to the lysosomal membrane, where it is activated in response to amino acid signaling. In turn, mTORC1 suppresses lysosomal activity by phosphorylating TFEB. T cells from old adults had lower lysosomal gene expression and proteolytic activities due to reduced TFEB transcription. In parallel, mTORC1 activity was more sustained in activated T cells from older adults. How lysosome-deficient aged T cells maintain mTORC1 activity remains unresolved. The late endosome is an alternative platform for mTORC1 activation. Compared to the lysosome, late endosomes have no proteolytic activity and therefore no amino acid efflux. They originate by homotypic fusion and vacuole protein sorting (HOPS) complex-mediated conversion from early endosomes. Inhibiting early to late endosomal conversion by silencing Vam6/Vps39-like protein (VPS39, a key member of the HOPS complex) led to the formation of hybrid endosomal compartments and attenuated mTORC1 activity. Conversely, inhibition of lysosomal activities induced an intracellular expansion of late endosomes. Consistent with this observation, an increased number of late endosomes is seen in responses of T cells from older individuals that fail to replenish their lysosomes.

### Study Design

The aim of this study was to examine the mechanism how activated naïve CD4 T cells from older adults exhibit increased and sustained mTORC1 activation in spite of lysosomal dysfunction, and to identify targets for intervention other than direct mTORC1 inhibition to improve T follicular helper cell responses and memory T cell generation. Purified naïve CD4 T cells collected from young and old healthy individuals were used to perform in vitro signaling and functional studies after polyclonal activation. Pharmacological inhibition as well as gene expression silencing were applied to interrogate lysosomal and endosomal pathways in the context of age. In vitro human data were validated in vivo in an infection mouse model by adoptively transferring antigen-specific CD4 T cells that had been genetically manipulated. Mice were randomly assigned to control versus sample groups. Date analysis was conducted in an unblinded manner. Sampling and experimental replicates were specified in figure legends. No outliers were removed.

### Study Population and Cell Isolation

PBMC were collected from 53 young (20-35 years old) and 64 old (65-85 years old) healthy individuals with no history of autoimmune disease or cancer and no uncontrolled renal disease, diabetes mellitus, or cardiovascular disease. 98 of them were de-identified samples purchased from the Stanford Blood Center (Palo Alto, CA, USA) from donors younger than 35 years (43 donors) or older than 65 years (55 donors). 19 samples were from individuals recruited from the local area. The study was in accordance with the Declaration of Helsinki, approved by the Stanford Institutional Review Board, and all participants gave informed written consent. Naïve CD4 T cells were purified with human CD4 T cell enrichment cocktail (15062, STEMCELL Technologies), followed by negative selection with anti-CD45RO magnetic beads (19555, STEMCELL Technologies).

### Cell Culture

Isolated human naïve CD4 T cells were activated with Dynabeads Human T-Activator CD3/CD28 (11132D, Thermo Fisher Scientific) in RPMI 1640 (Sigma) supplemented with 10% fetal bovine serum (FBS) and 100 U/mL penicillin and streptomycin (Thermo Fisher Scientific). Mouse T cells were activated in plates coated with 8 μg/mL anti-CD3 Ab (16-0032-82, eBioscience) and 8 μg/mL anti-CD28 Ab (16-0281-82, eBioscience) in culture medium supplemented with 10 ng/ml IL-2 (21212, Peprotech).

### Western Blotting

Cells were lysed in RIPA buffer containing PMSF and protease and phosphatase inhibitors (sc-24948, Santa Cruz Biotechnology) for 30 minutes on ice. Proteins were separated on denaturing 4%-15% SDS-PAGE (4561086, Bio-Rad), transferred onto nitrocellulose membrane (1704270, Bio-Rad) and probed with antibodies to SLC7A5 (5347S), c-MYC (18583S), β-actin (4970S), S6K1 (2708S), pS6K1 Kinase (Thr389, 9234S), EEA1 (3288S), RAB7 (9367S), Cathepsin D (2284S), Na/K ATPase (3010S), Tubulin (2125S), mTOR (2983S), Raptor (2280S), RHEB (13879S), PD-1 (86163S, all Cell Signaling Technology), CD63 (ab68418), Cathepsin H (ab185935) and VPS39 (ab224671, all Abcam). Membranes were developed using HRP-conjugated secondary antibodies (Cell Signaling Technology) and Chemiluminescent Western Blot Detection Substrate (Thermo Fisher Scientific).

### Flow Cytometry

For cell surface staining, cells were incubated with fluorescently conjugated antibodies in PBS containing 2% FBS at 4° C. for 30 minutes. For intracellular cytokine assays, cells were stimulated with 10 UM LCMV GP66-77 DIYKGVYQFKSV (SEQ ID NO:5) or 0.2 μM LCMV GP33-41 KAVYNFATC (SEQ ID NO:6) (Anaspec) in the presence of Brefeldin A (GolgiPlug, BD Biosciences) for 4 hours at 37° C. Cells were then incubated with antibodies to cell surface molecules, permeabilized with Cytofix/Cytoperm kit (BD Biosciences) and stained with fluorescently labeled antibodies specific for the indicated cytokines. For pS6RP (S235/S236) staining, cells were treated with FOXP3 Fix/Perm Buffer Set (421403, Biolegend) before incubation with fluorescently conjugated antibodies at room temperature for 60 minutes. Dead cells were excluded from the analysis using LIVE/DEAD Fixable Aqua (eBioscience). Staining for flow cytometry was done with monoclonal antibodies against: CD4 (anti-human: RPA-T4; anti-mouse: RM4-5), CD8 (anti-human: RPA-T8; anti-mouse: 53-6.7), CD44 (IM7), B220 (RA3-6B2), Fas (Jo2), GL-7 (GL7), CD138 (281-2), IgD (11-26), CD62L (MEL-14), CD127 (SB/199), KLRG-1 (2F1), pS6RP (S235/S236, N7-548, all BD Bioscience), PD-1 (anti-human: A17188B; anti-mouse: 29F.1A12), CTLA-4 (UC10-4B9), IFN-γ (XMG1.2), TNF-α (MP6-XT22) and IL-2 (JES6-5H4, all Biolegend). Mouse CXCR5 was stained with biotin-conjugated anti-CXCR5 (2G8, BD Bioscience) followed by APC-streptavidin binding (BD Bioscience). D^{b} GP33-41 KAVYNFATC (GP33) tetramer was obtained from the NIH tetramer core facility (Atlanta, GA). To stain LCMV-specific CD8 T cells, cells were incubated with D^{b} GP33 tetramers along with cell surface antibodies at 4° C. for 30 minutes in antibody staining buffer. Cells were analyzed on an LSRII or LSR Fortessa (BD Biosciences). Flow cytometry data were analyzed using FlowJo (TreeStar).

### RNA Isolation and Quantitative RT-PCR

Total RNA was isolated using the RNeasy Plus Mini Kit (74134, QIAGEN) and converted to cDNA using the SuperScript VILO cDNA Synthesis Kit (11754, Invitrogen). Quantitative RT-PCR was performed on the ABI 7900HT system (Applied Biosystems) using Power SYBR Green PCR Master Mix (4368706, Thermo Fisher Scientific), according to the manufacturer's instructions. Oligonucleotide primer sets are shown in Table 1.

TABLE 1
Oligonucleotide primer sets used in this study.

| Gene | Forward | SEQ ID NO | Reverse | SEQ ID NO |
|---|---|---|---|---|
| SLC7A5 | CCGTGAACTGCTACAGCGT | 7 | CTTCCCGATCTGGACGAAGC | 8 |
| SLC7A1 | GTCCTGCTCAACATTGGGCA | 9 | CAGGGCCTGCATTCTCACG | 10 |
| PDCD1 | CCAGGATGGTTCTTAGACTCCC | 11 | TTTAGCACGAAGCTCTCCGAT | 12 |
| CTSB | CCAGGGAGCAAGACAGAGAC | 13 | GAGACTGGCGTTCTCCAAAG | 14 |
| CTSD | GACACAGGCACTTCCCTCAT | 15 | CTCTGGGGACAGCTTGTAGC | 16 |
| CTSH | ACGGAGGAGTACCACCACAG | 17 | GCAATTCTGAGGCTCTGACC | 18 |
| CTSS | TCTCTCAGTGCCCAGAACCT | 19 | GCCACAGCTTCTTTCAGGAC | 20 |
| TFEB | GACCCAGAAGCGAGAGCTCACA | 21 | TGTGATTGTCTTTCTTCTGCCG | 22 |
| 18S rRNA | CGCCGCTAGAGGTGAAATTCT | 23 | CGAACCTCCGACTTTCGTTCT | 24 |

### Gene Set Enrichment Analysis

Gene set enrichment analysis (GSEA) software from the Broad Institute (software.broadinstitute.org/gsea/index.jsp) was used to determine the enrichment of gene sets in T cells from young (20-35 years) or old (65-85 years) adults. The datasets describing age-associated differences in activated CD4 T cells were obtained from SRA database under accession number SRP158502. See, e.g., Hu, et al., Aging Cell 18, e12957 (2019).

### Transfection

Naive CD4 T cells were transfected with either SMARTpool negative control siRNA, SMARTpool TFEB siRNA, SMARTpool MYC siRNA or SMARTpool VPS39 siRNA (all from Dharmacon) using the Amaxa Nucleofector system and P3 primary cell Nucleofector Kit (Lonza). Cells were rested for 2 hours, washed and activated by dynabeads for 5 days. Cells were then harvested and analyzed.

### Pharmacologic Inhibition

The SLC7A5 inhibitor JPH203 (S8667, Selleckchem) was used at a dose of 5 μM for all experiments. Lysosome inhibitors Chloroquine (PHR1258-1G, Sigma-Aldrich) and Bafilomycin A1 (11038, Cayman CHEMICAL) were used at doses of 20 μM and 10 nM, respectively. The mTORC1 inhibitor Torin 1 (4247, Tocris) was used at a dose of 100 nM. The protein synthesis inhibitor cycloheximide (C4859, Sigma-Aldrich) was used at a dose of 5 μg/mL.

Endosome Isolation and In Vitro mTORC1 Kinase Activity Assay

Endosomes were isolated from naïve CD4 T cells on day 3 after activation when mTORC1 activity peaked, by using an endosome isolation kit (ED-028, Invent Biotechnologies) according to manufacturer's instructions. In some experiments, cells were pre-treated for 2 hours with 1 μM AKT inhibitor (MK-2206 2HCl, Selleckchem) prior to harvesting. Briefly, 2×10⁷ cells were suspended in lysis buffer A (supplemented with protease inhibitor cocktail) and cell extracts were filtered to remove intact cells, larger organelles and plasma membranes. The flow-through cell lysates were mixed with the supplied precipitation buffer B followed by centrifugation. After centrifugation, endosomes were enriched in pellets. Supernatants were discarded, and the endosome pellets were resuspended in RIPA lysis buffer (sc-24948, Santa Cruz Biotechnology). Protein concentrations were measured using BCA Protein Assay Kit (23227, Pierce) and equal amounts of protein were loaded for immunoblotting analysis of indicated markers. Alternatively, the endosome pellets were resuspended in mTORC1 kinase buffer (25 mM HEPES, 50 mM KCl, 10 mM MgCl₂, 20% glycerol, 4 mM MnCl₂ and 250 μM ATP) prepared according to a previous study (Kim, et al., Cell 110, 163-175 (2002)) and divided into aliquots of 10 μL each containing 10 μg endosomes. 100 ng of S6K1 Human Recombinant Protein (TP317324, Origene) diluted in 5 μL of mTORC1 kinase buffer was added as the substrate, and the Kinase assays were performed at 30° C. for 20 minutes in a final volume of 15 μL. Reactions were stopped by adding 5 μL of 4× sample buffer and loaded for immunoblotting analysis.

### Confocal Microscopy

Cells were fixed in 4% paraformaldehyde, permeabilized with 0.1% Triton X-100, and incubated with primary antibodies to LAMP1 (#9091), mTOR (#2983), pS6RP (S235/236, #4858) and EEA1 (#48453, all Cell Signaling Technology) at 4° C. overnight. Incubation with secondary antibodies was performed at room temperature for 2 hours using Alexa Fluor 488-conjugated AffiniPure Donkey anti-Rabbit immunoglobulin G (IgG) or Cy3-conjugated AffiniPure Donkey anti-Mouse IgG (Jackson Immuno Research Laboratories). The images were analyzed using an LSM 710 microscope system with ZEN 2010 software (Carl Zeiss) and a 63× oil immersion objective (Carl Zeiss).

### DQ-BSA Lysosomal Activity Assay

Cells were treated with 5 μg/mL of DQ-BSA (D12050, Thermo Fisher Scientific) diluted in prewarmed medium and incubated at 37° ° C. for 6 hours. Cells were then briefly washed once with ice-cold PBS containing 2% FBS and kept on ice. Fluorescence of cleaved BSA-DQ was analyzed by flow cytometry.

### Cell Proliferation Assay In Vitro

Freshly purified naïve CD4 T cells were labeled with CellTrace Violet (CTV; Thermo Fisher Scientific) and stimulated with anti-CD3/anti-CD28 beads in the presence of recombinant human PD-L1/B7-H1 Fc chimera (156-B7, R&D systems; 5 μg/mL) crosslinked by goat anti-human IgG Fc polyclonal antibody (G-102-C, R&D systems; 5 μg/ml). At day 5-6, cells were harvested and analyzed by flow cytometry.

### SARS-CoV-2 and Pertussis-Reactive CD4 and CD8 T Cell Responses

PBMCs were isolated by density gradient centrifugation using Lymphoprep (STEMCELL Technologies) from pheresis samples of SARS-CoV-2 unexposed healthy donors. The HLA class II peptide megapool covering the entire SARS-CoV-2 orfeome and the HLA class I peptide megapool covering half of the entire orfeome were described previously. See, Grifoni, et al., Cell Host Microbe, 27:671-680-e672 (2020); Grifoni, et al., Cell, 181:1489-1501 e1415 (2020). PBMCs were labeled with CellTrace Violet (CTV; Thermo Fisher Scientific) and cultured with either the mixture of SARS-CoV-2 class II megapool (1 μg/mL) and class I megapool (1 μg/mL) or pertussis megapool (1 μg/mL) in RPMI 1640 media (Sigma) supplemented with 5% human AB serum (Sigma), 1 μg/mL anti-CD28 (ebioscience) and 100 U/mL penicillin and streptomycin (Thermo Fisher Scientific). VPS39-silencing FANA ASO (FANA antisense oligonucleotide, 1 μM, AUM Biotech), scrambled control FANA ASO (1 μM, AUM Biotech), anti-PD-1 blocking antibody (A17188B, 1 μg/mL) or control IgG (1 μg/mL) were added to the culture on day 0. Peptide-reactive CD4 and CD8 T cell responses were measured on day 8.

### Mice, Adoptive Transfers and LCMV Infection

Naïve CD4 T cells specific to the GP66-77 epitope of LCMV obtained from 5- to 8-week-old SMARTA TCR transgenic mice (CD45.1, provided by Rafi Ahmed at Emory University) were activated in plates coated with anti-CD3 Ab and anti-CD28 Ab. Cells were transduced with a retroviral vector expressing either scrambled control RNA, Tfeb shRNA (5′-CGGCAGTACTATGACTATGAT-3′ (SEQ ID NO:25)), Vps39 shRNA (5′-AGTGAGCATGTGCTGAAGAAG-3′ (SEQ ID NO:26)) or Sle7a5 shRNA (5′-CGCAATATCACGCTGCTCAAC-3′ (SEQ ID NO:3)) on days 1 and 2 after activation. On day 6 after activation, retrovirus-transduced Amcyan-positive SMARTA cells were isolated and 1×10⁴ transduced cells were intravenously (i.v.) transferred to 5- to 8-week-old naive C57BL/6 (CD45.2) mice (Jackson Laboratory). On day 3 post-transfer, mice were infected intraperitoneally (i.p.) with 2×10⁵ PFU of LCMV Armstrong. On day 8 post-infection, spleens were harvested and analyzed. For PD-1 Blockade: anti-PD-1 (29F.1A12, #BE0273, Bio X Cell; 200 μg, i.p.) or control IgG (2A3, #BE0089, Bio X Cell; 200 μg, i.p.) was administered to LCMV-infected B6 mice on days 0, 3 and 6 post infection. On day 8 post-infection, spleens were harvested and analyzed. For recall responses of CD8 memory T cells, recombinant Listeria monocytogenes expressing the LCMV glycoprotein 33-41 epitope (Lm-gp33) were grown to log phase in BHI broth. Concentrations were determined by measuring the O.D. at 600 nm (O.D. of 1=1×10⁹ CFU/ml). LCMV-immune mice were injected i.v. with 2×10⁵ colony forming units (CFU) for recall responses. All animal experiments were approved by the Stanford Administrative Panel on Laboratory Animal Care Committee.

### ELISA

Serum was collected from mice on day 14 after LCMV (Armstrong) infection.

LCMV-specific IgG was measured using an anti-LCMV-NP IgG ELISA kit (AE-300200-1, Alpha Diagnostic International) according to the manual. Briefly, ELISA plates coated with LCMV-VP1 antigen were pre-washed and incubated with 5-fold serial diluted serum for 60 min. Plates were then washed and incubated with anti-mouse IgG HRP for 30 minutes. After washing, bound antibody was detected by adding TMB substrate and the reaction was stopped with stop solution. Plates were read for absorbance at 450 nm.

### Western Blotting

Cells were lysed in RIPA buffer containing PMSF and protease and phosphatase inhibitors (sc-24948, Santa Cruz Biotechnology) for 30 minutes on ice. Proteins were separated on denaturing 4%-15% SDS-PAGE (4561086, Bio-Rad), transferred onto nitrocellulose membrane (1704270, Bio-Rad) and probed with antibodies to AKT (4685S), pAKT (Ser473, 4060S), anti-mouse TFEB (32361S), anti-human p-TFEB (Ser211, 37681S), FOXO1 (2880T, all Cell Signaling Technology), anti-human TFEB (ab220695, Abcam) and VPS39 (ab224671, Abcam). Membranes were developed using HRP-conjugated secondary antibodies (Cell Signaling Technology) and Chemiluminescent Western Blot Detection Substrate (Thermo Fisher Scientific).

### TGF-β SMAD Reporter Assays

TGF-β/SMAD signaling activity were determined by using the SBE Reporter Kit (60654, BPSbioscience) according to manufacturer's instructions. Briefly, 1×10⁶ naïve CD4 T cells were cotransfected with 500 ng of reporter (component A) and either 0.4 nmol of control siRNA or 0.4 nmol of VPS39 siRNA. Alternatively, cells were cotransfected with 500 ng of negative control reporter (component B) and either 0.4 nmol of control siRNA or 0.4 nmol of VPS39 siRNA. Each condition was set up triplicates. After transfection, cells were stimulated with anti-CD3/SMAD signaling activity were determined by using the SBE Reporter Kitanti-CD28 dynabeads in the presence or absence of 10 ng/SMAD signaling activity were determined by using the SBE Reporter Kitml TGF-β. On day 3 after activation, cells were analyzed for luciferase activities by using the Dual-Luciferase® Reporter Assay System (Promega). The normalized luciferase activity was obtained by subtracting background luminescence from the negative control followed by calculating the ratio of firefly luminescence from the SBE reporter to Renilla luminescence from the control Renilla luciferase vector.

### Statistical Analysis

Statistical analysis was performed using the Prism 7.0 software (GraphPad Software Inc.). Paired or unpaired two-tailed Student's t-tests were used for comparing two groups. One-way ANOVA with Tukey's post hoc test was used for multi-group comparisons. p<0.05 was considered statistically significant. Statistical details and significance levels can be found in the figure legends.

### Example 2—Lysosome-Independent Activation of mTORC1 in Naïve CD4 T Cell Responses

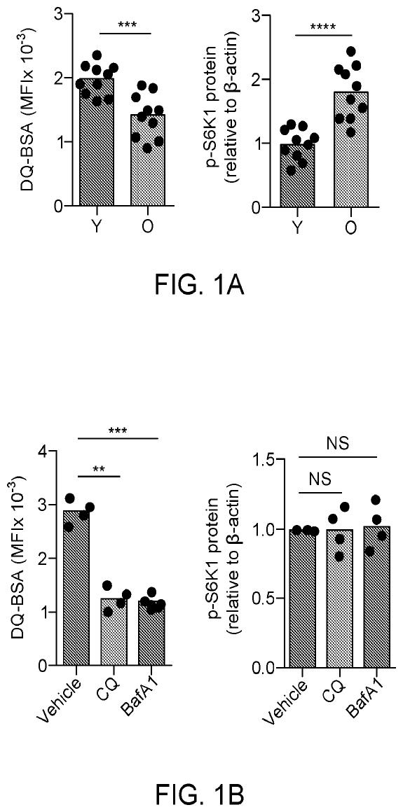

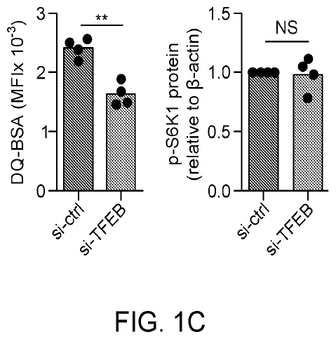

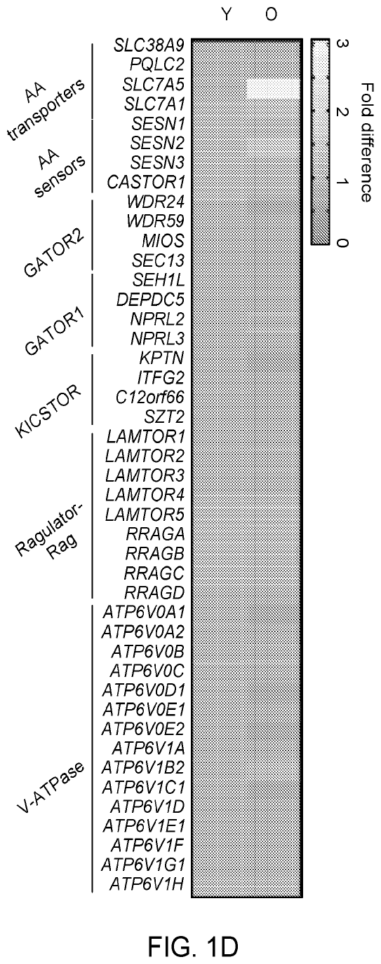

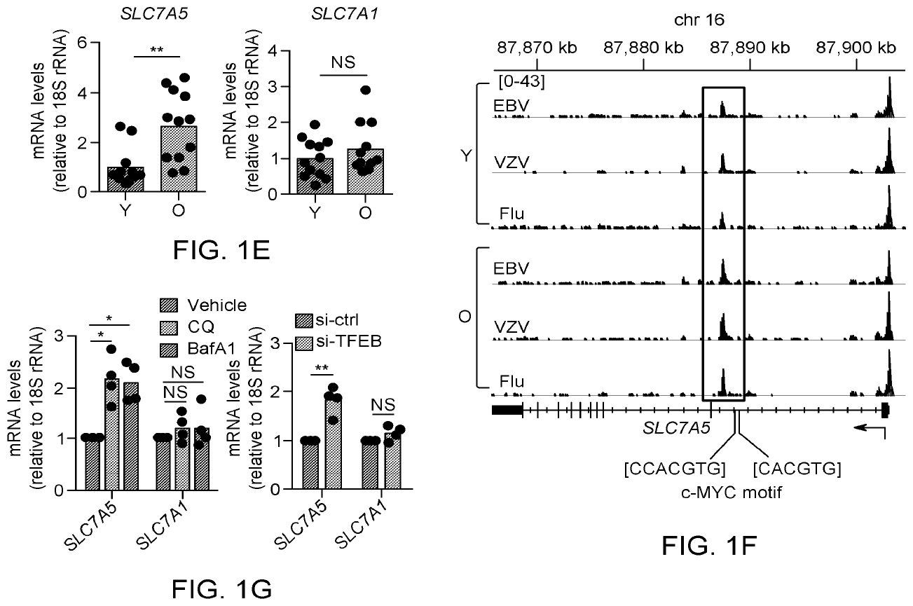

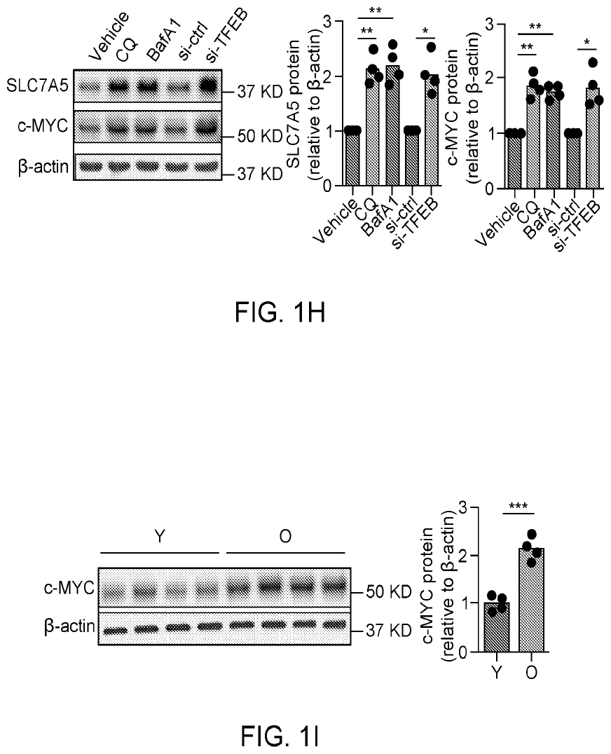

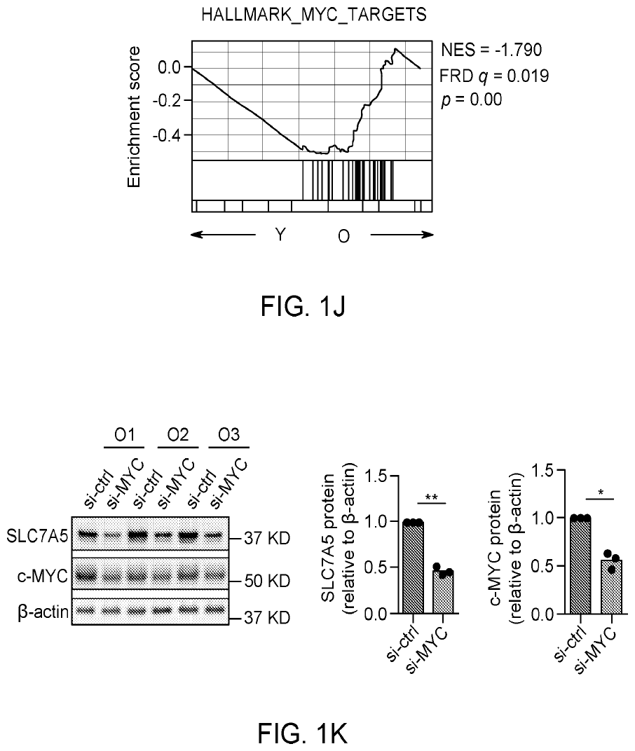

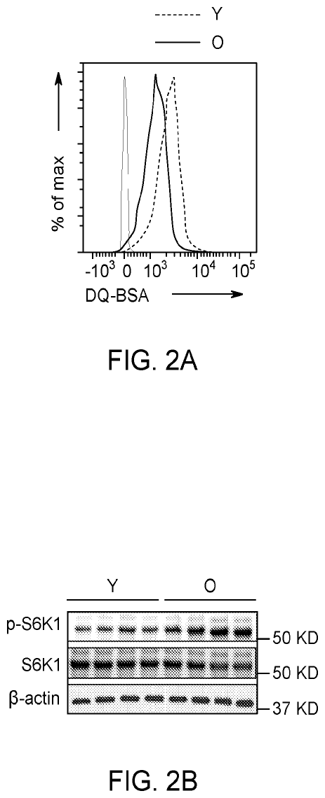

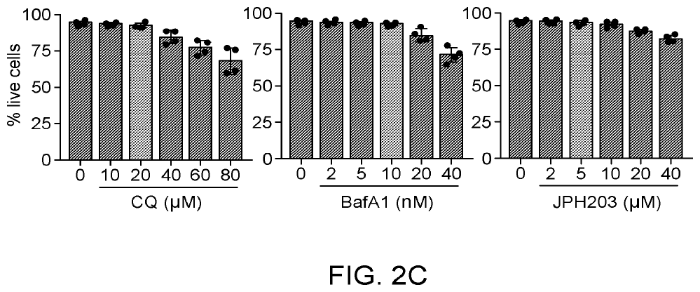

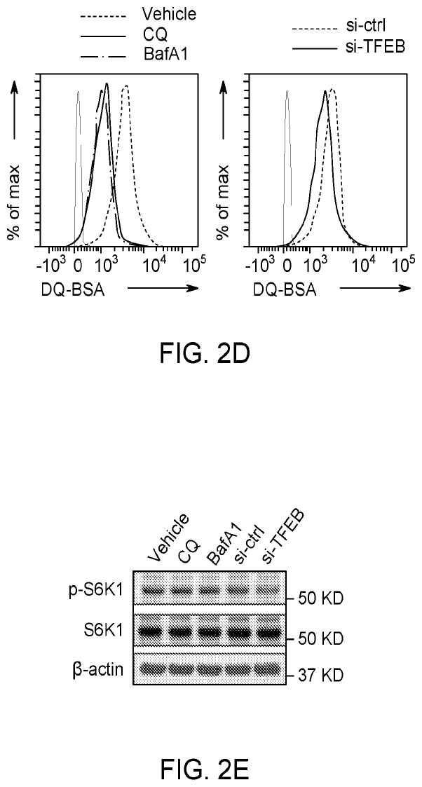

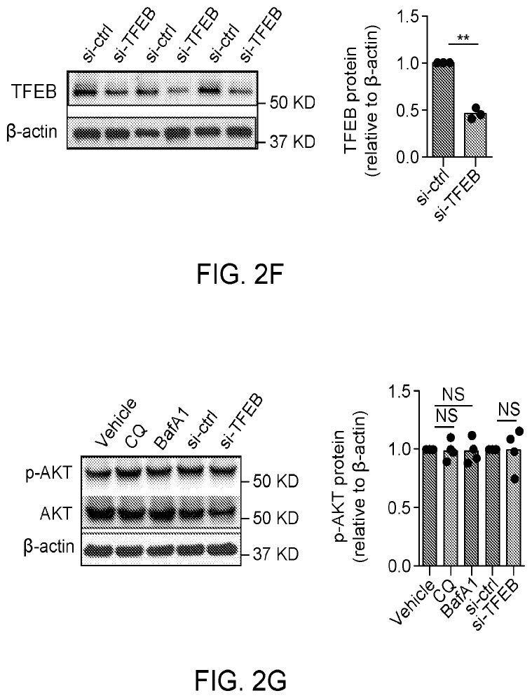

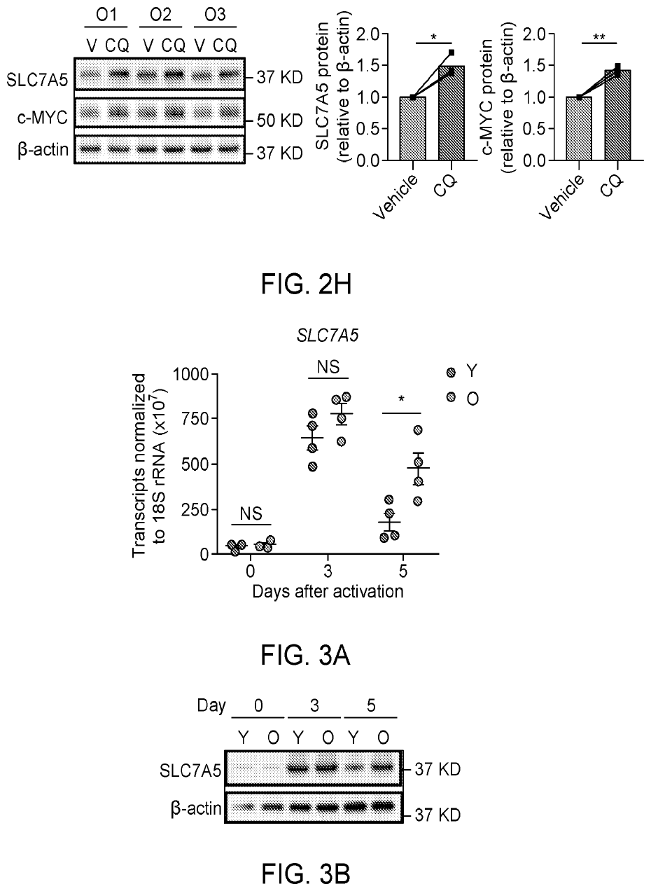

mTORC1 is recruited to lysosomal membranes where it is activated by released amino acids. However, day 5-activated naïve CD4 T cells from old adults have impaired lysosomal activity in parallel to enhanced mTORC1 activity as shown by reduced fluorescence of cleaved de-quenched bovine serum albumin (DQ-BSA) and increased S6K1 protein phosphorylation, the downstream effector of mTORC1 activity (FIG. 1A and FIGS. 2A and B). To examine this apparent conundrum, the effects of lysosome inhibition was investigated on mTORC1 activity; chloroquine and Bafilomycin A1, two lysosomal acidification inhibitors, were used for pharmacological inhibition at nontoxic doses (FIG. 2C); TFEB silencing was used as a genetic intervention. All interventions reduced lysosomal activities but surprisingly neither reduced S6K1 (FIGS. 1B and C and FIG. 2D to F) nor AKT phosphorylation (FIG. 2G), the downstream effector of the growth-factor signaling arm of mTORC1 pathway. See, for example, Liu, et al., Nat Rev Mol Cell Biol 21, 183-203 (2020).

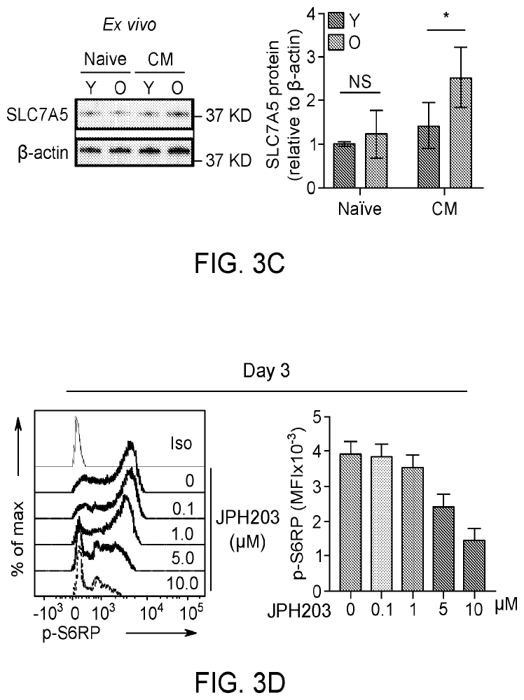

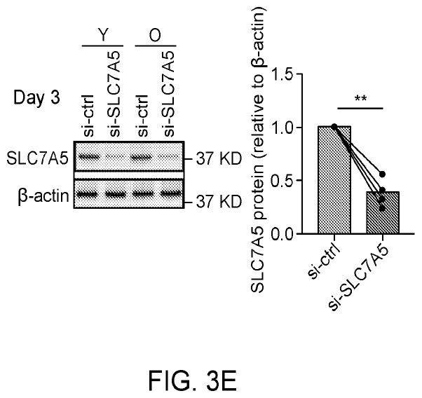

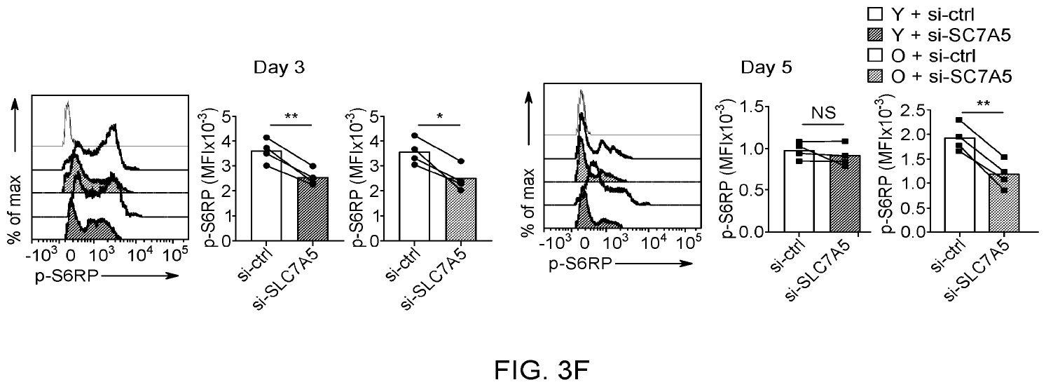

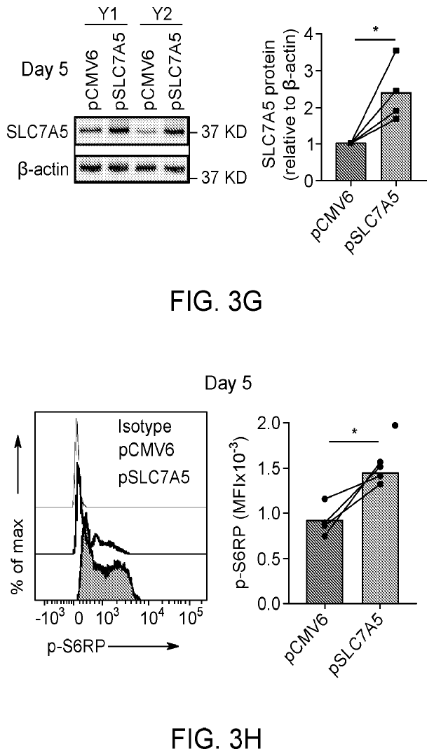

The mechanism was investigated of how mTORC1 activities is even increased under lysosome-deficient conditions in T cell responses of old adults. Because lysosome deficiency likely affects lysosomal efflux of amino acids, expression of genes encoding the amino acid signaling arm of the mTORC1 pathway was examined using data from a recent RNA-seq study comparing naïve CD4 T cell responses from young and old adults after activation. See, Hu, et al., 2019, supra. SLC7A5, which encodes a plasma membrane leucine transporter, is selectively more expressed in T cells from old adults while other genes involved in the nutrient arm do not change with age (FIG. 1D). Increased SLC7A5 transcription was confirmed in an independent cohort of day 5-activated naïve CD4 T cells from twelve young and twelve old individuals. In contrast, no difference was seen for SLC7A1 that encodes the arginine transporter (FIG. 1E). SLC7A5 expression increased with activation in naïve CD4 T cells from young and old donors, but expression was more sustained beyond day 3 in old naïve CD4 T cells (FIG. 3A-B). This difference persisted and was still seen for memory CD4 T cells (FIG. 3C). Accordingly, ATAC-seq studies showed increased chromatin accessibility of SLC745 in old memory CD4 T cells in three virus-specific (EBV, VZV and influenza) systems compared to young cells (FIG. 1F). Consistent with increased SLC7A5 expression in lysosome-deficient aged T cells, pharmacological inhibition of lysosome activity or genetic silencing of TFEB upregulated SLC7A5 transcript and protein expression (FIGS. 1G and H and FIG. 2H). These data suggested that the up-regulation of SLC745 transcription in day 5-stimulated naïve CD4 T cells from old adults was mechanistically related to their reduced lysosome activity.

Upstream of the differentially opened chromatin sites of SLC7A5 are two c-MYC binding motifs (FIG. 1F). c-MYC is the major driver of SIC7A5 transcription in T cell responses. See, Marchingo, et al., Elife 9, (2020). As shown in FIG. 1H, c-MYC protein expression was increased after lysosomal inhibition or TFEB silencing, indicating a lysosome-dependent degradation of c-MYC protein. Consistently, day 5-activated aged naïve CD4 T cells had higher c-MYC protein level compared to young cells (FIG. 1I). Global gene expression profiles obtained by RNA-seq supported the notion that day 5-activated old naïve CD4 T cells had higher c-MYC activity. Gene set enrichment analysis (GSEA) showed that age-associated transcriptional signatures of day 5-activated naïve CD4 T cells were correlated with the expression pattern of genes in the Hallmark_MYC_target pathway (FIG. 1J). To confirm that the increased SLC7A5 expression is related to higher c-MYC protein expression, c-MYC silencing was performed in old naïve CD4 T cells and found reduced SLC7A5 protein expression (FIG. 1K). Taken together, these data showed that lysosome deficiency stabilized c-MYC protein, thereby increasing SLC7A5 expression in day 5-stimulated aged naïve CD4 T cells

### Example 3—SLC7A5-Dependent Late Endosomal mTORC1 Activation in Naïve CD4 T Cell Responses

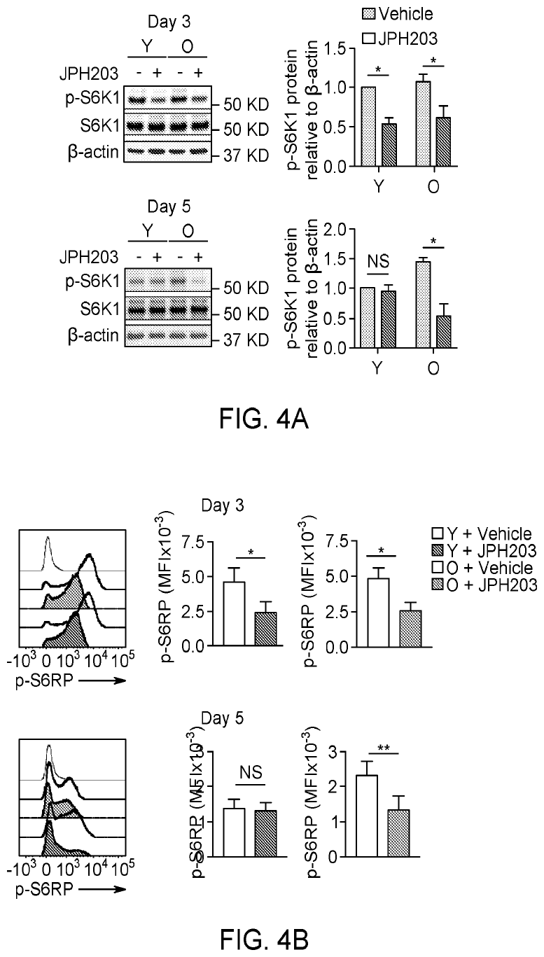

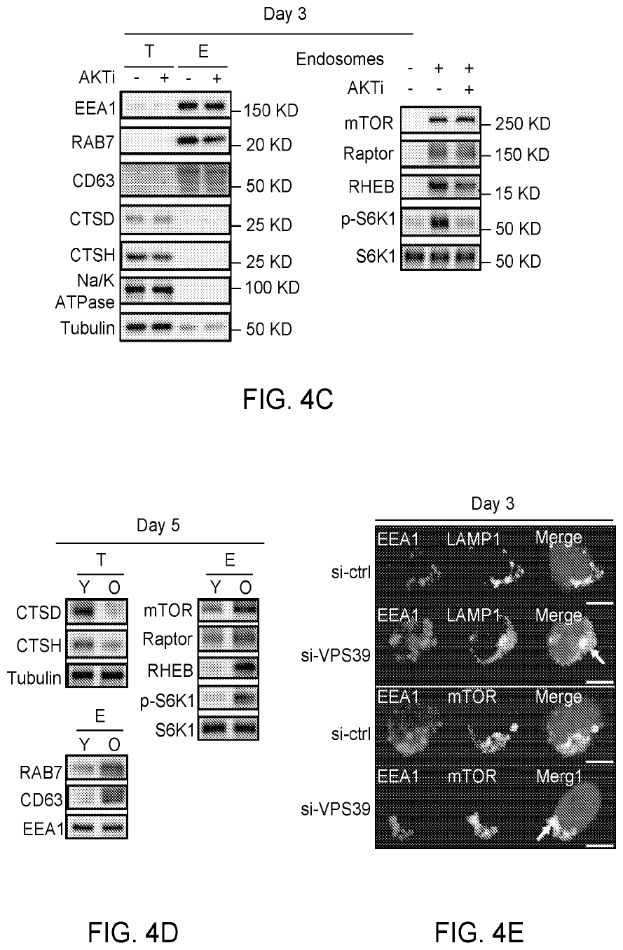

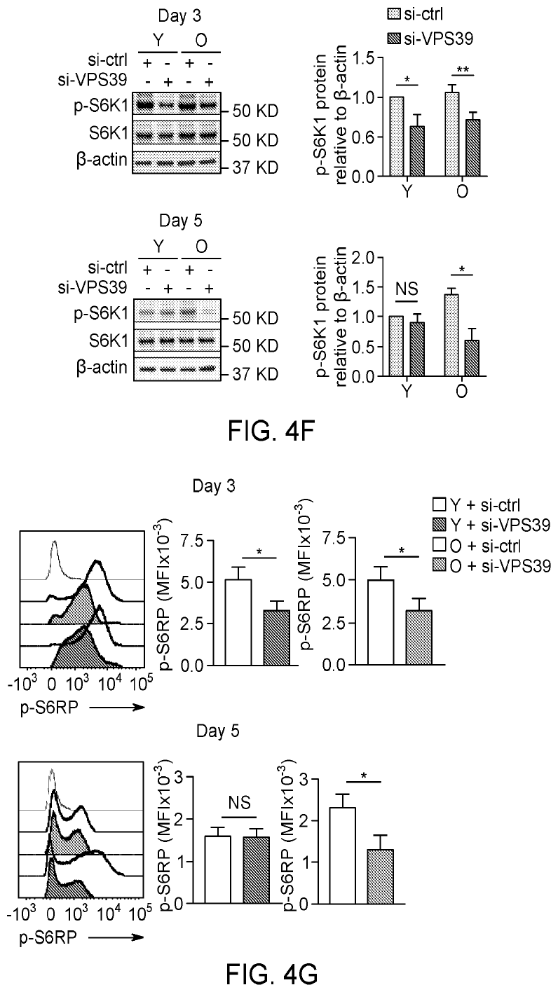

To determine whether SLC7A5 activity accounts for the sustained mTORC1 activity in day 5-stimulated old naïve CD4 T cells, T cell cultures from young and older adults were treated with the specific SLC7A5 inhibitor JPH203. mTORC1 activities were monitored by Western blotting of S6K1 phosphorylation or by flow cytometry of S6RP phosphorylation on days 3 and 5 after activation. In titration experiments, a dose of 5 μM JPH203 was not cytotoxic (FIG. 2C) and inhibited mTORC1 activity by about 50% (FIG. 3D). On day 3, SLC7A5 inhibition reduced mTORC1 activity in both young and old cells (FIGS. 4A and B). On day 5, mTORC1 activity was already attenuated in T cell responses of young adults. In contrast, mTORC1 activity remained high in old activated T cells; the excess activity was sensitive to SLC7A5 inhibition (FIGS. 4A and B). To exclude off-target effects of the inhibitor, genetic SLC7A5 silencing and overexpression were performed. Data observed were consistent with those obtained by JPH203 treatment (FIG. 3E-H). Taken together, old activated CD4 T cells had a sustained uptake of extracellular amino acids through SLC7A5 to maintain mTORC1 activity in spite of reduced lysosomal function.

Activated T cells from old adults failed to rebuilt their lysosomes and expand the late endosomal compartments. Jin, et al., Sci Adv 6, eaba1808 (2020). To investigate whether these late endosomes provide an alternative platform for mTORC1 signaling in T cells, endosome fractions were isolated from naïve CD4 T cells on day 3 after stimulation when mTORC1 activity peaks in both young and old individuals. The endosome isolates were enriched for early and late endosome markers (EEA1, RAB7 and CD63) and depleted of the lysosome markers Cathepsin D and Cathepsin H, and of the plasma membrane marker Na/K ATPase, indicating the high purity of the endosome extracts (FIG. 4C). These endosome isolates contained mTOR, Regulatory-associated protein of mTOR (Raptor) and Ras homolog enriched in brain (RHEB) that are essential for substrate recruitment and kinase activity of mTORC1 (FIG. 4C). In an in vitro kinase assay, these endosome isolates exhibited mTORC1 kinase activity toward the substrate S6K1 that was not seen in endosomes extracted from cells treated with an AKT inhibitor (FIG. 4C). Day 5-stimulated naïve CD4 T cells from old individuals had reduced lysosomal cathepsins, similar early endosome contents (EEA1) and increased late endosomal compartments, with increased late endosomal mTOR, Raptor and RHEB protein levels and higher mTORC1 kinase activity as shown by increased S6K1 phosphorylation in the in vitro kinase assay (FIG. 4D). Taken together, these data indicate that mTORC1 is activated on late endosomes and that late endosomal compartments are expanded in stimulated naïve CD4 T cells from old adults accounting for the sustained mTORC1 activity.

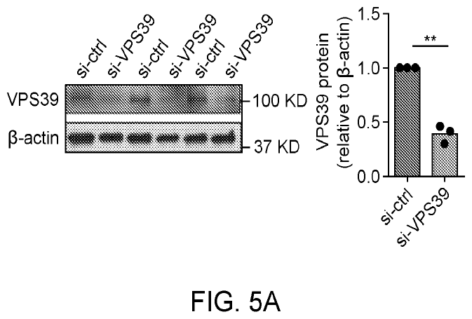

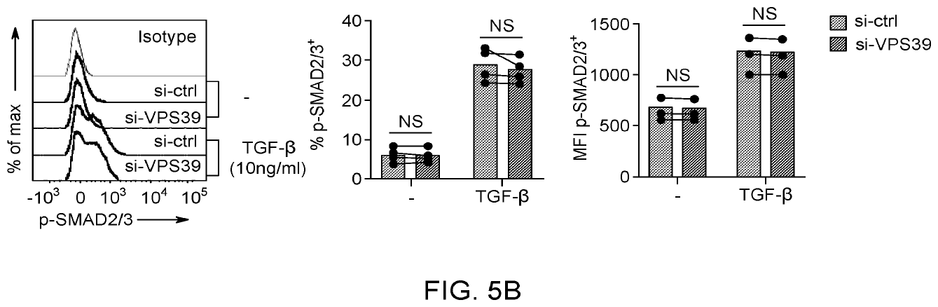

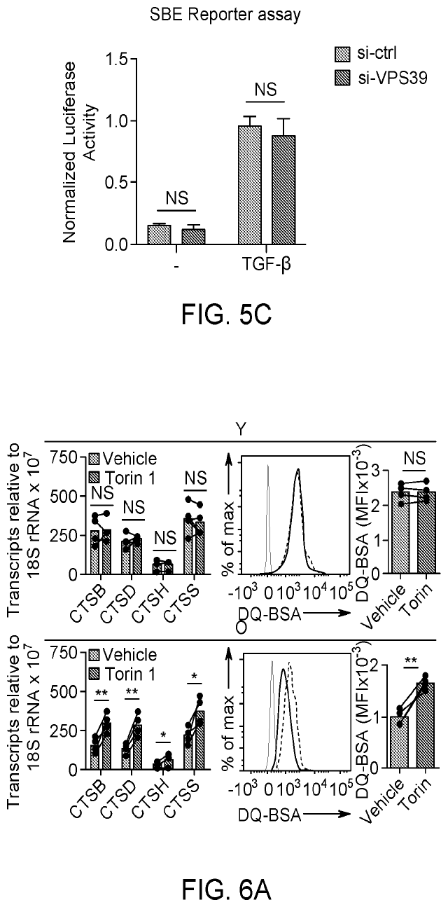

VPS39 silencing was performed to examine whether inhibition of late endosome maturation prevents mTORC1 activation. Confocal imaging confirmed that VPS39 silencing inhibited late endosome maturation and produced hybrid endosomal compartments that contain both the early endosome marker EEA1 and the late endosome/lysosome marker LAMP1 (FIG. 4E). These hybrid compartments co-localized with mTOR (FIG. 4E); however. S6K1 and S6RP phosphorylation were reduced after VPS39 silencing documenting that these hybrid structures do not support mTORC1 activation (FIGS. 4F and G). T cells from young and old adults exhibited reduced mTORC1 activities after VPS39 silencing on day 3 after T cell stimulation. On day 5, an effect of silencing was only observed in T cells from old adults (FIGS. 4F and G) suggesting that the sustained mTORC1 signaling observed with age depended on the formation of late endosomes. VPS39 has also been implicated in regulating TGF-β/SMAD signaling (see, e.g., Felici, et al., EMBO J 22, 4465-4477 (2003)); however, no changes of SMAD2/3 phosphorylation or reporter activities of a SMAD reporter were observed in T cells after VPS39 silencing (FIG. 5A-C), supporting the notion that the observed reduced mTORC1 activity is due to its impact on endosome maturation and not SMAD signaling.

### Example 4—Sustained Activation of Late Endosomal mTORC1 Suppresses Lysosomal Activities in Naïve CD4 T Cell Responses

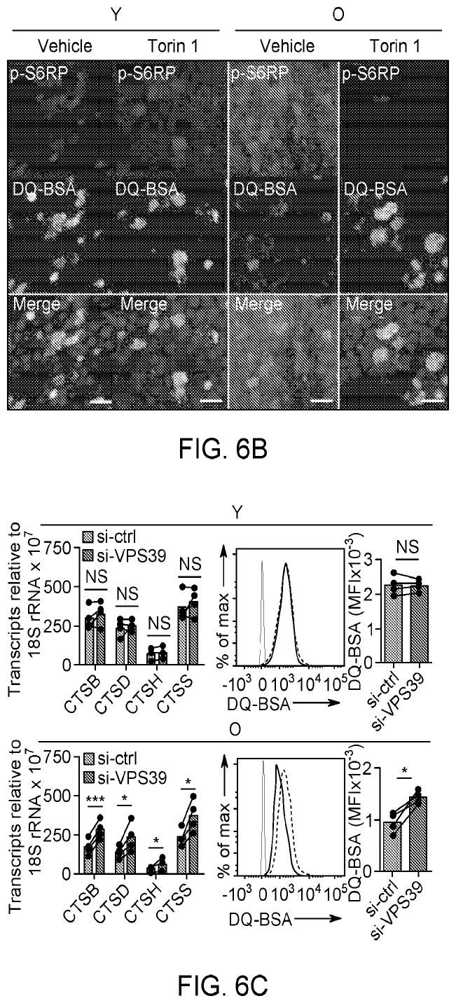

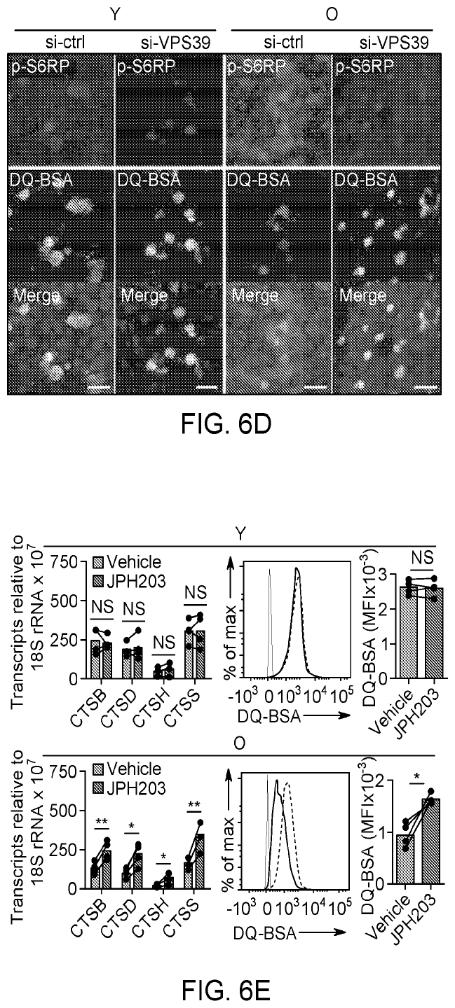

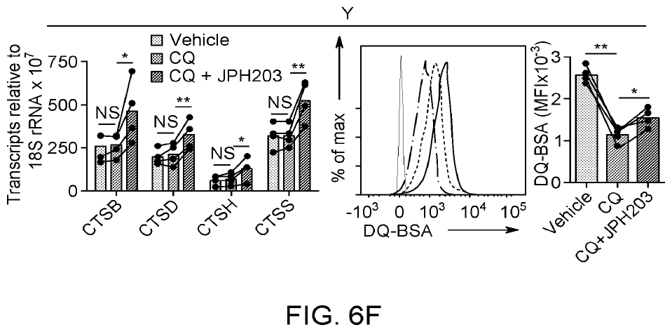

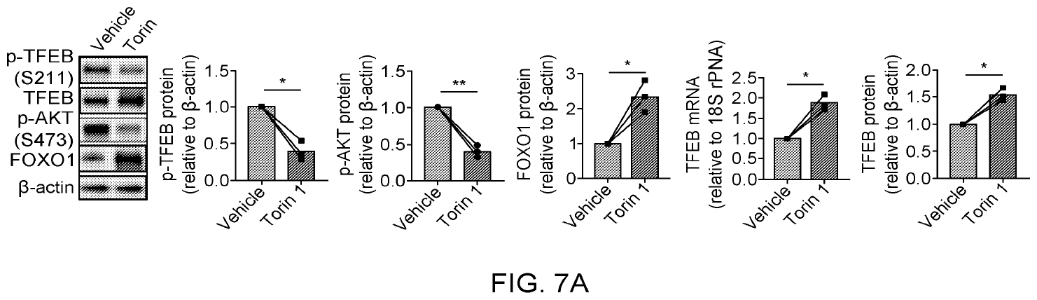

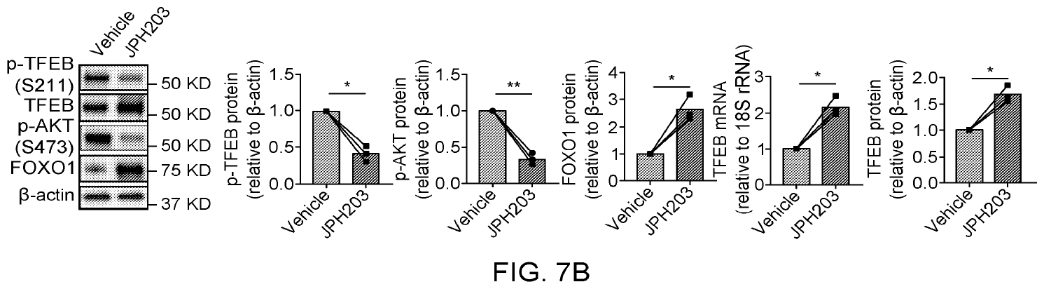

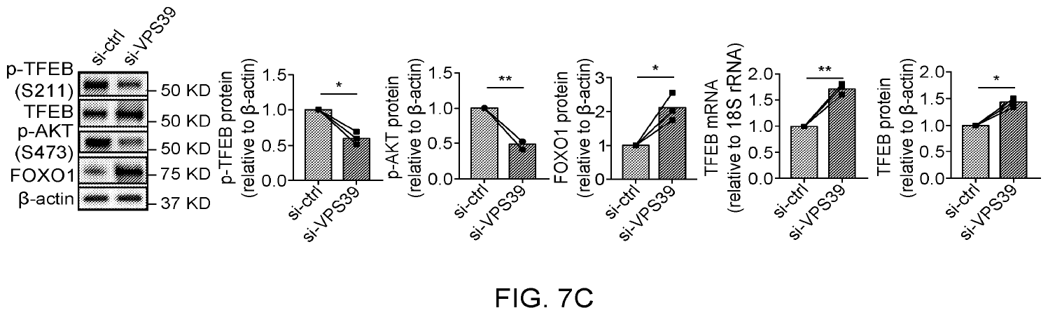

The data thus far demonstrated that CD4 T cells from old adults sustain mTORC1 activity on late endosomes that relies on the presence of SLC7A5 and VPS39. mTORC1 phosphorylates TFEB, thereby preventing its transcriptional activity and inhibiting lysosome function. See, e.g., Roczniak-Ferguson, et al., Sci Signal 5, ra42 (2012). mTORC1 inhibition downregulated TFEB phosphorylation while upregulating TFEB mRNA and protein levels due to reduced AKT-mediated FOXO1 protein degradation (FIG. 7A-C). Consistent with this notion, mTORC1 inhibition by Torin 1 of day 5-activated T cells from old adults partially restored the TFEB-dependent transcription of lysosomal cathepsin genes ((TSB, CTSD, CTSH and CTSS) (see, e.g., Jin, et al., Sci. Adv., 6 eaba 1808 (2020)) and lysosomal proteolytic activities (FIGS. 6A and B). Inhibition of late endosomal mTORC1 in activated T cells from old adults by either VPS39 silencing or SLC7A5 inhibition had similar effects, while the already higher lysosomal activity in T cells from young adults could not be further increased (FIG. 6C-E). However, SLC7A5 inhibition could counteract the effects of lysosome inhibition in young T cells. As shown in FIG. 6F, lysosomal cathepsins and lysosomal activities were increased in CQ and JPH203-cotreated young cells compared to young T cells treated with CQ alone. Together, these data demonstrate that sustained activation of mTORC1 in late endosomes suppressed lysosomal activities in day 5-stimulated naïve CD4 T cells from old adults.

### Example 5—Sustained Activation of Late Endosomal mTORC1 Prevents PD-1 from Lysosomal Degradation

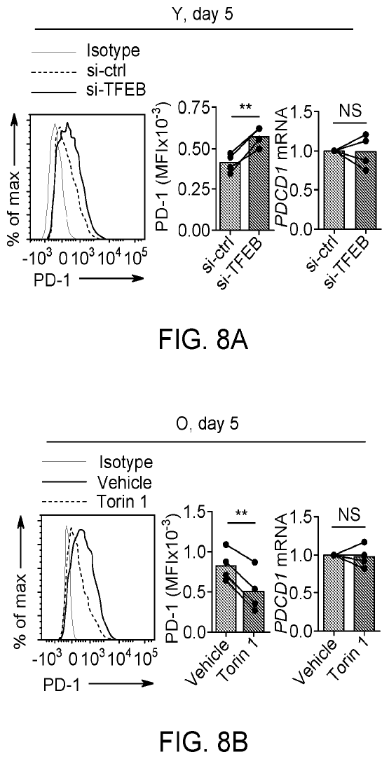

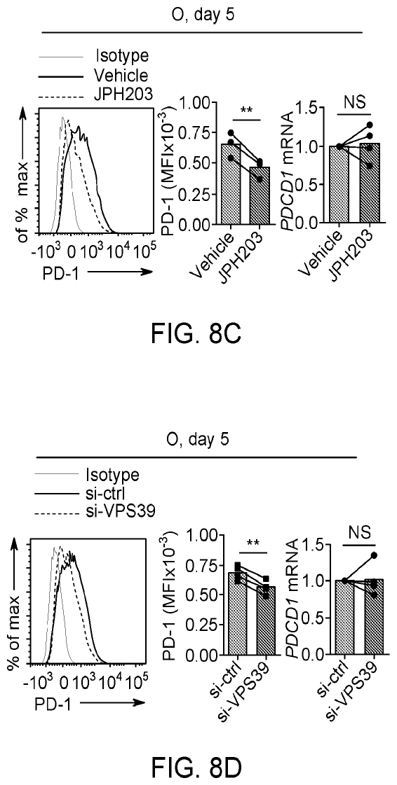

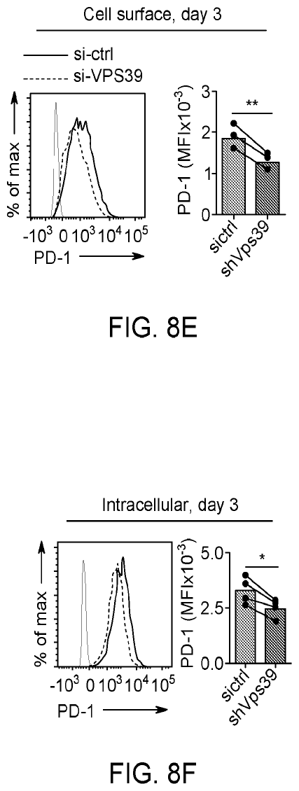

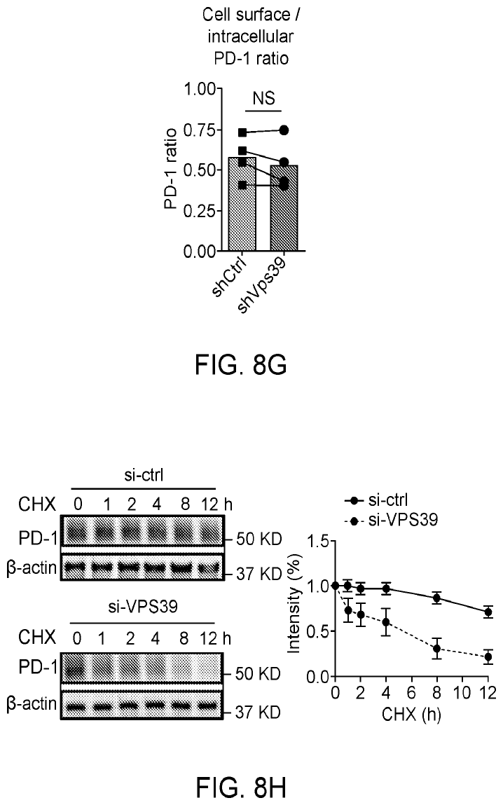

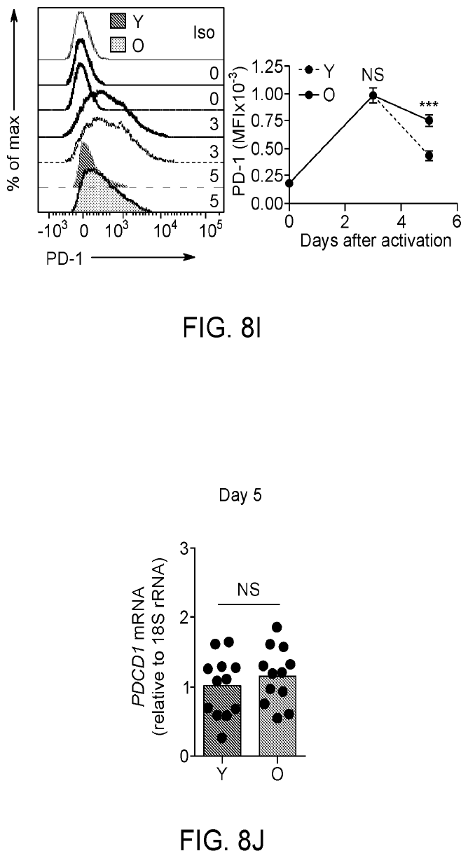

It was previously observed that in the course of T cell proliferation after activation FOXO1 promoted the generation of lysosomes through induction of TFEB transcription. See, e.g., Jin, et al., Sci. Adv. 6, eaba 1808 (2020). Moreover, Foxo1-deficient mouse CD4 T cell showed up to fourfold increase of PD-1 protein levels after antigen priming in vivo (Stone, et al., Immunity 42, 239-251 (2015)), raising the possibility that lysosomal activity regulates PD-1 protein expression. As shown in FIG. 4A, lysosome inhibition by TFEB silencing in T cells from young adults increased cell surface protein expression of PD-1 while transcript levels remained unchanged. In contrast, stimulation of lysosomal activities following late endosomal mTORC1 inhibition in T cells from old adults by either Torin 1 treatment, SLC7A5 inhibition or VPS39 silencing reduced cell surface protein expression without effects on transcripts of PD-1 (FIG. 8B-D). This effect was not due to the changes of intracellular vs cell surface trafficking routes of PD-1 since VPS39 silencing equally reduced cell surface and intracellular PD-1 protein expression (FIG. 8E-G). Assessment of PD-1 half-life in vitro in day 5-stimulated old naïve CD4 T cells confirmed that VPS39-silenced cells underwent enhanced PD-1 protein degradation than control-silenced cells (FIG. 8H). These data suggest that mTORC1 at late endosomes stabilizes PD-1 protein by preventing lysosomal degradation of PD-1.

To determine whether PD-1 protein regulation changes with age, PD-1 expression was monitored at days 0, 3 and 5 after stimulation by flow cytometry. Consistent with the changes of the kinetics of mTORC1 activity with age, PD-1 protein expression peaked at day 3 after activation in both young and old activated naïve CD4 T cells to then subside in activated T cells from young but not old adults. As shown in FIGS. 8I and J, there were no age-related differences of protein expression of PD-1 at days 0 or 3, but activated T cells from old adults had more PD-1 protein at day 5 while no difference in PDCD1 transcription.

### Example 6—Sustained Activation of Late Endosomal mTORC1 Impairs Expansion of NaïV CD4 T Cells from Old Adults

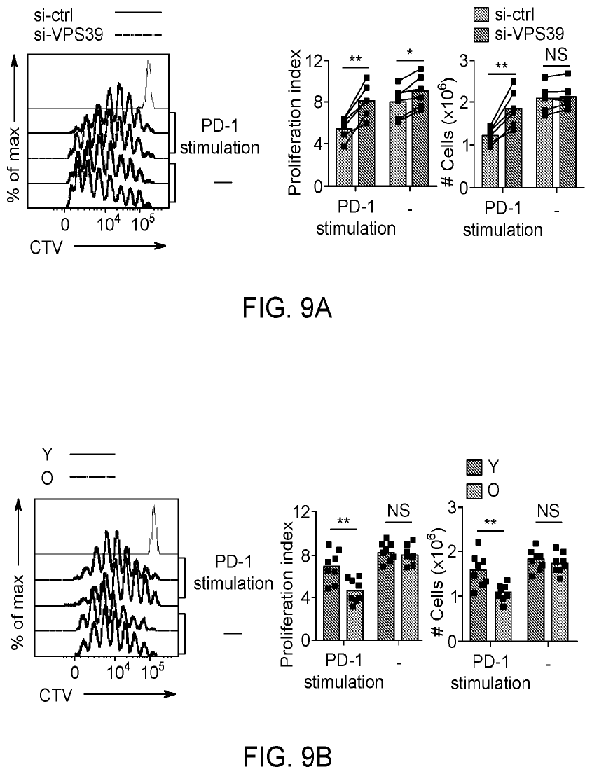

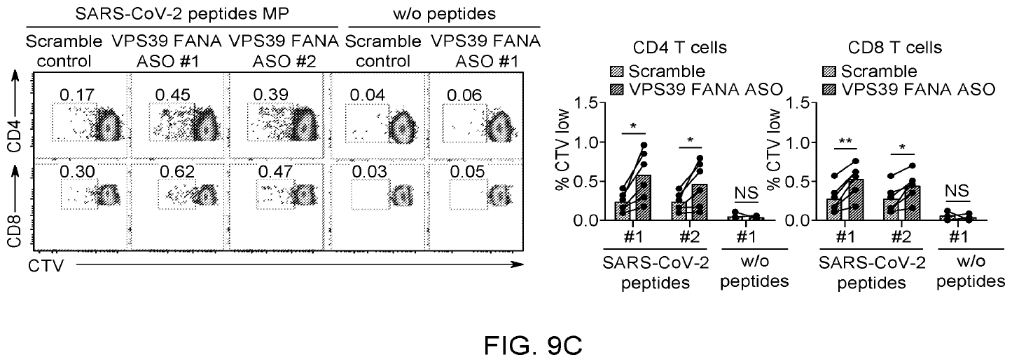

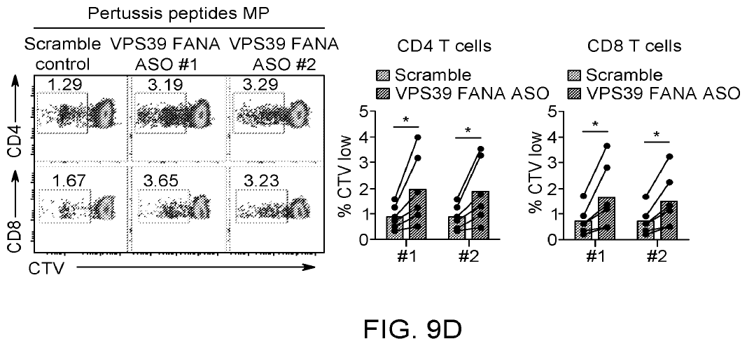

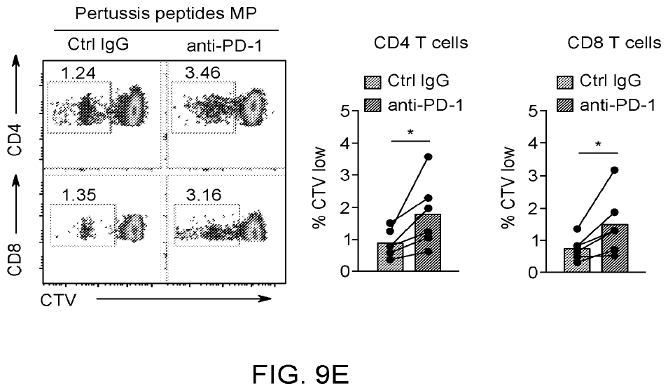

Failure to timely degrade PD-1 protein raises the possibility that lysosome-deficient T cells receive an increased inhibitory signal suppressing cell expansion. To test this hypothesis, naïve CD4 T cells were stimulated with anti-CD3/anti-CD28 beads in the presence or absence of PD-L1-Fc and anti-human IgG to cross-link PD-1 (Shi, et al., Immunity, 49, 264-274 e264 (2018)). VPS39 silencing induced increased proliferation and cell recovery in the presence of PD-1 stimulation (FIG. 9A). In contrast, VPS39 silencing only caused a minor increase in CD4 T cell proliferation in the absence of PD-1 crosslinking that was not sufficient to increase cell numbers (FIG. 9A). These data indicate that late endosomal mTORC1 activity regulates T cell expansion through lysosomal degradation of PD-1. Consistent with their sustained mTORC1 activation on late endosomes and reduced lysosomal activity and therefore degradation of PD-1, naïve CD4 T cells from old adults had reduced potential to proliferate and to expand in the presence of PD-1 crosslinking (FIG. 9B). In the absence of exogenous PD-L1-Fc, age-related proliferation differences were not observed supporting the notion that the proliferative defect was related to increased PD-1 expression in T cells from old adults (FIG. 9B).

A similar effect of endosomal differentiation interference was also seen for antigen-specific T cell responses. In these experiments, CTV-labeled PBMCs from healthy individuals were stimulated with a pool containing both HLA class II peptide megapool covering the entire SARS-CoV-2 orfeome together with HLA class I peptide megapools covering half of the entire orfeome. To silence VPS39 in human T cells, FANA antisense oligonucleotide (FANA ASO) was added, which has the advantage of easy self-delivery without the need of transfection regents. Two VPS39 FANA ASO clones (#1 and #2) were added to replicate cultures at day 0. Frequencies of divided T cells were determined on day 8. Among eleven SARS-CoV-2-unexposed individuals tested, CD4 and CD8 T cell responses to SARS-CoV-2 peptide pool were observed in six individuals. VPS39 FANA ASO clone #1 and #2 significantly increased the SARS-CoV-2-reactive CD4 and CD8 T cell expansion compared to scrambled control FANA ASO in those individuals who showed a response (FIG. 9C). A similar increase of proliferating cells was not seen for PBMCs cultured in the absence of antigenic peptides (FIG. 9C). Consistent with recent reports that SARS-CoV-2 responses in unexposed individuals derive from memory T cells, VPS39 silencing improved human T cell responses to pertussis peptide pools (FIG. 9D). A similar increase in pertussis-specific responses was seen with anti-PD-1 blockade (FIG. 9E).

### Example 7—Inhibition of Late Endosomal mTORC1 Promotes Primary CD4 T Cell Responses after LCMV Infection In Vivo

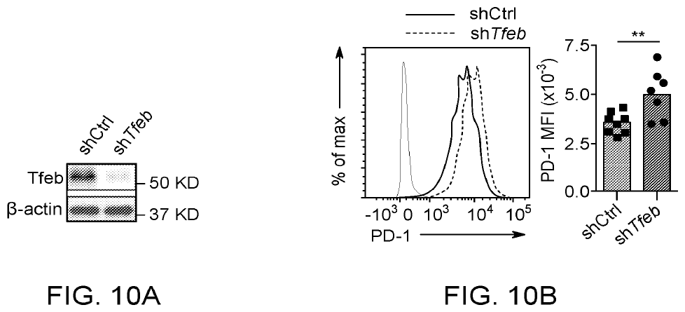

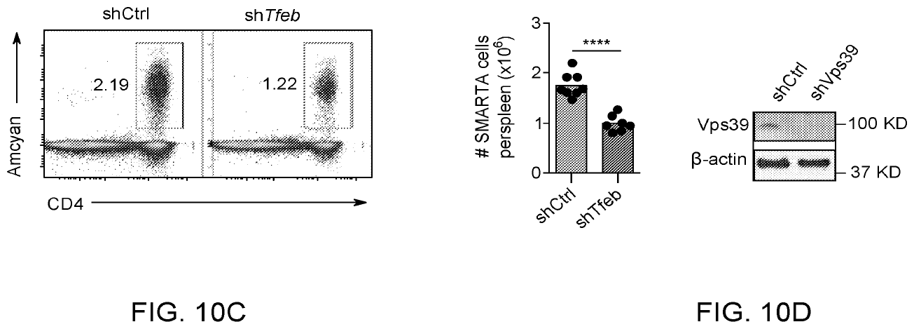

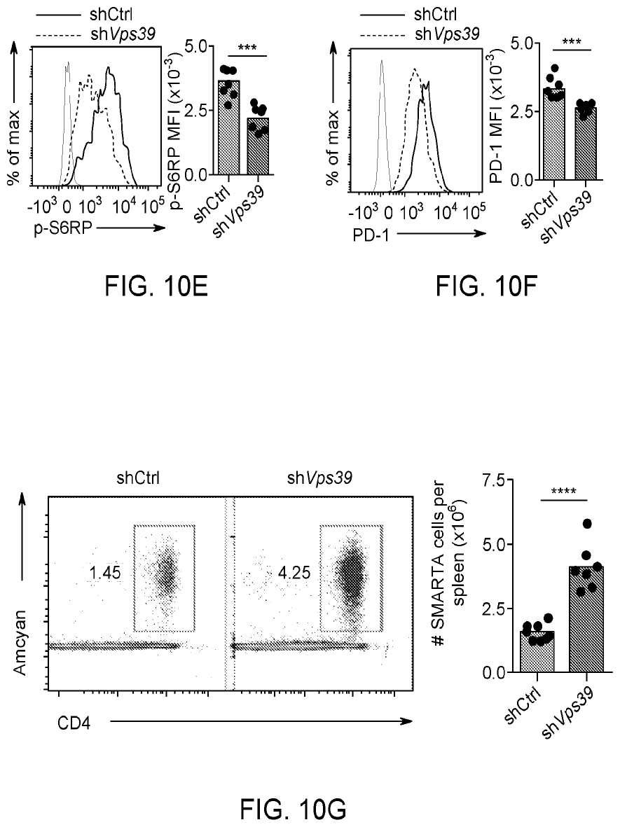

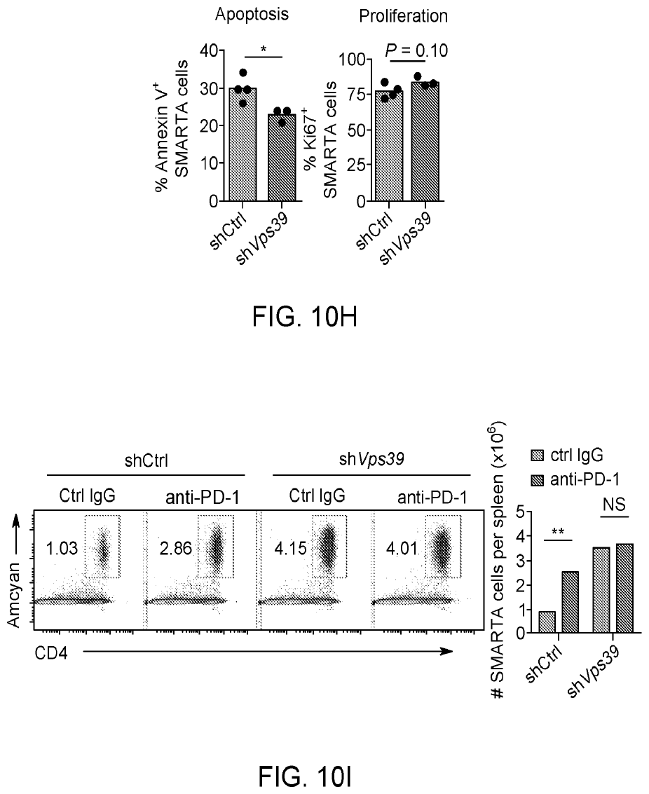

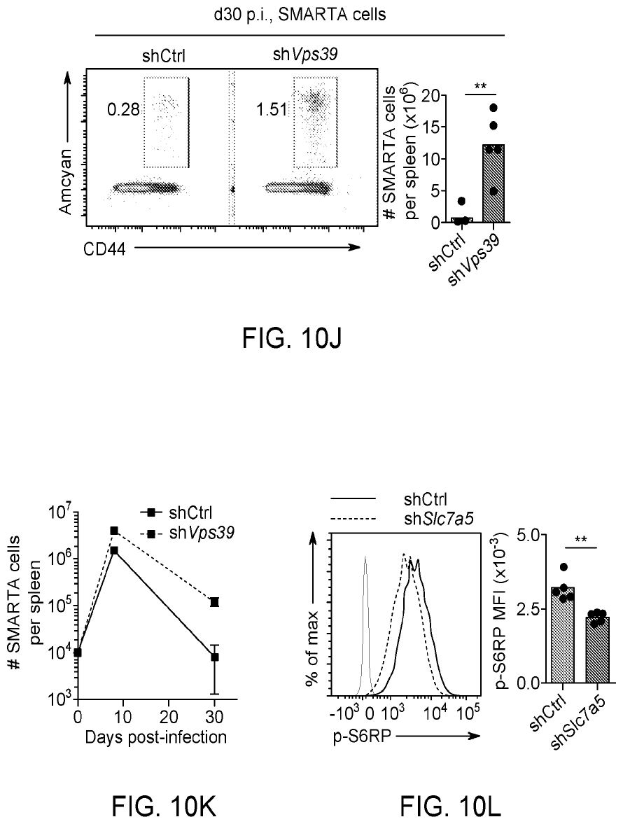

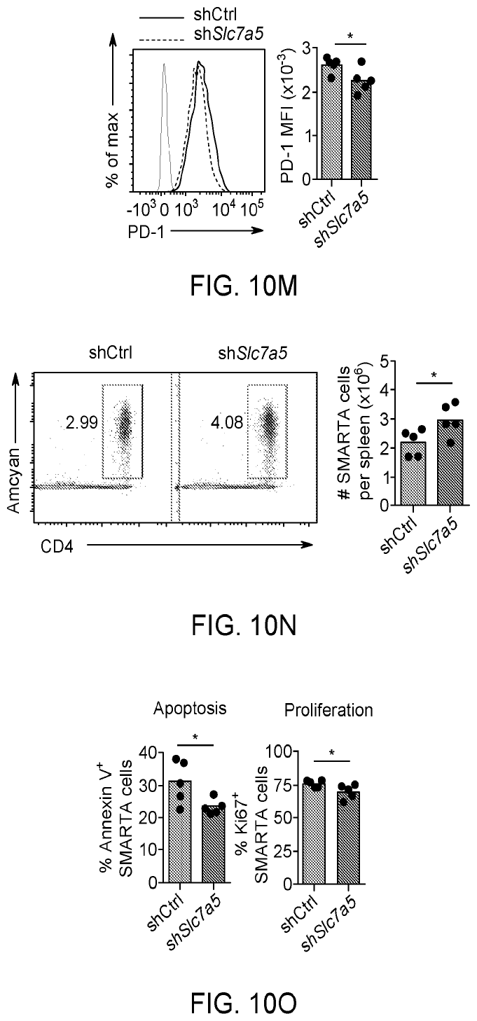

To determine whether improved expansion due to lysosome dysfunction is also seen with antigen-specific T cell responses in vivo, the LCMV infection mouse model was used. SMARTA CD4 T cells were retrovirally transduced with shRNA specific for Tfeb (shTfeb) or control shRNA (shCtrl) and transferred the cells into 5- to 8-week-old B6 mice followed by acute LCMV infection. Tfeb silencing induced an increased PD-1 expression, leading to a reduced SMARTA CD4 T cell expansion at peak responses (FIG. 10A-C).

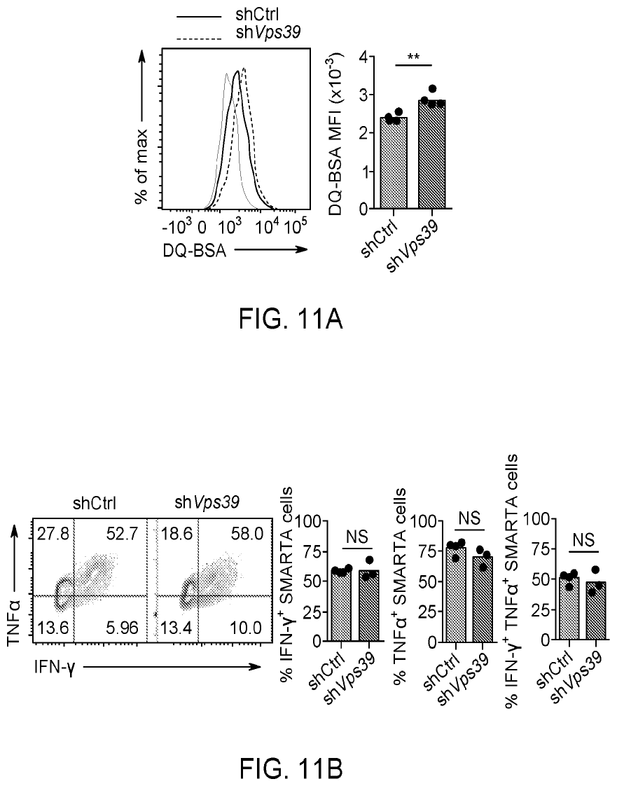

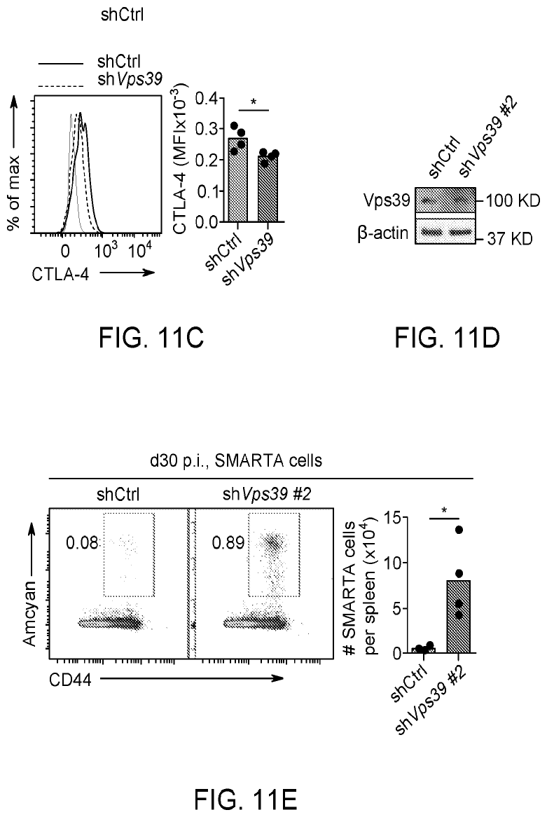

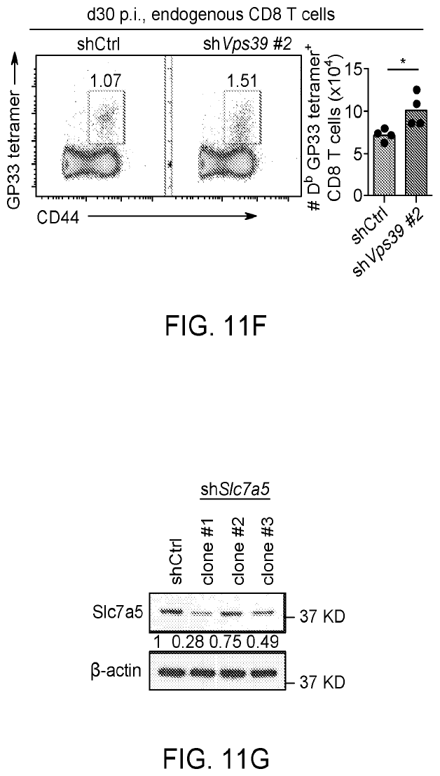

To determine whether LCMV-specific CD4 T cell responses can be boosted by inhibiting late endosomal mTORC1 in vivo, we transferred SMARTA CD4 T cells transduced with shRNA specific for Vps39 (shVps39) or control shRNA (shCtrl) into B6 mice followed by acute LCMV infection. Vps39-silenced SMARTA CD4 T cells had reduced mTORC1 activities, decreased PD-1 expression, enhanced lysosomal activities and up to a threefold increase in cell numbers in the spleen at peak responses on day 8 (FIGS. 10D-G and FIG. 11A). Vps39-silenced, expanded SMARTA CD4 T cells were functional and produced similar levels of cytokines as control-silenced cells after peptide restimulation ex vivo (FIG. 11B). Decrease of the Annexin V⁺ population indicated reduced apoptosis as a mechanism of the increased SMARTA CD4 T cell recovery after Vps39 silencing, with an additional trend for increased cell proliferation as shown by Ki67⁺ population (FIG. 10H). To further examine whether the increased SMARTA CD4 T cell expansion is at least in part due to reduced PD-1 expression, the PD-1/PD-L1 pathway was blocked by injecting mice with anti-PD-1 blocking antibody. As shown in FIG. 10I, PD-1 blockade promoted control-silenced SMARTA CD4 T cell expansion at peak responses after LCMV infection while it did not further improve Vps39-silenced SMARTA CD4 T cell expansion. At day 30 after infection, Vps39-silenced SMARTA CD4 T cells showed over 15-fold increase in cell numbers with less contraction during the effector/memory transition than control-silenced SMARTA cells (FIGS. 10J and K), consistent with the previously reported role of mTORC1 inhibition in promoting memory generation. See Araki, et al., Nature 460, 108-112 (2009)). Vps39 silencing by a second shRNA reproduced the data (FIGS. 11D and E), excluding the possibility of off-targets effects. Late endosomal mTORC1 inhibition by partial Slc7a5 silencing in SMARTA CD4 T cells (FIG. 11G) reproduced the data with Vps39 silencing, although to a lesser extent. mTORC1 activities and PD-1 protein expression were both reduced after Sle7a5 silencing, while SMARTA cell number were slightly increased (FIG. 10L-N). Again, this increase of cell number was mostly due to reduced cell apoptosis, while proliferation was even slightly decreased (FIG. 10O). Taken together, these data show that inhibition of late endosomal mTORC1 promotes virus-specific CD4 T cell expansion and memory generation after primary acute LCMV infection.

### Example 8—Inhibition of Late Endosomal mTORC1 Augments CD4 T Cell Helper Responses In Vivo

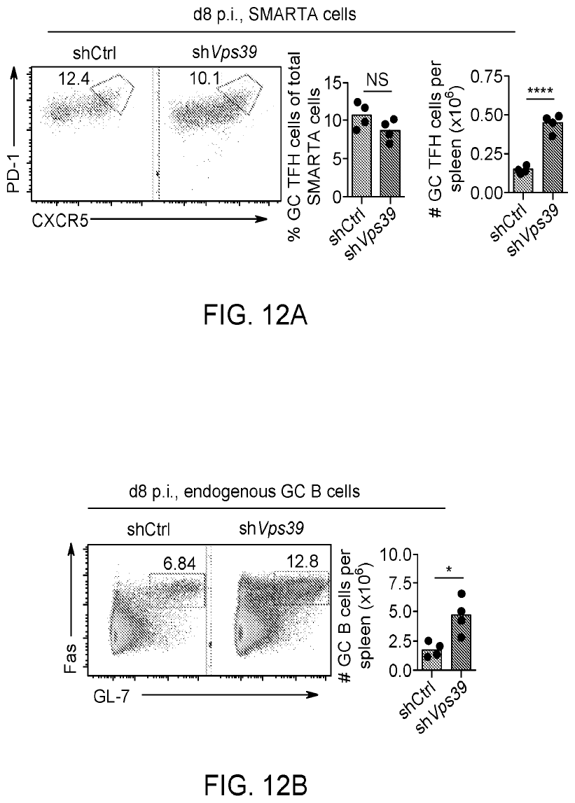

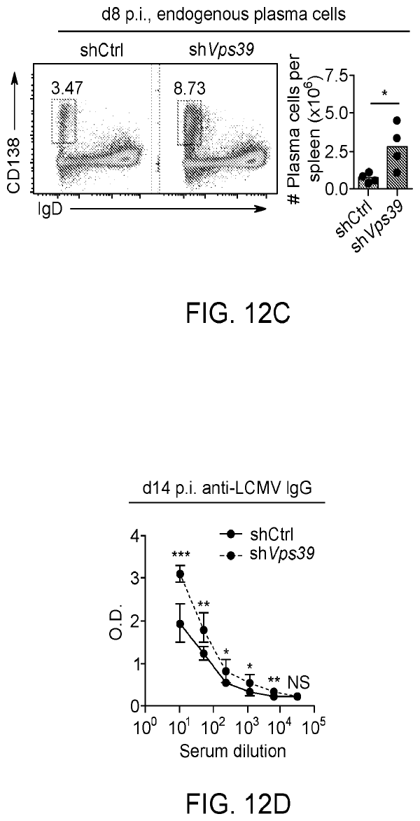

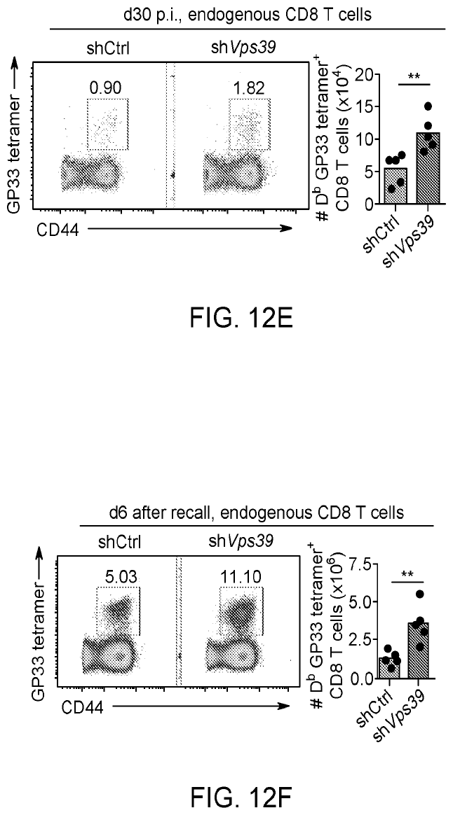

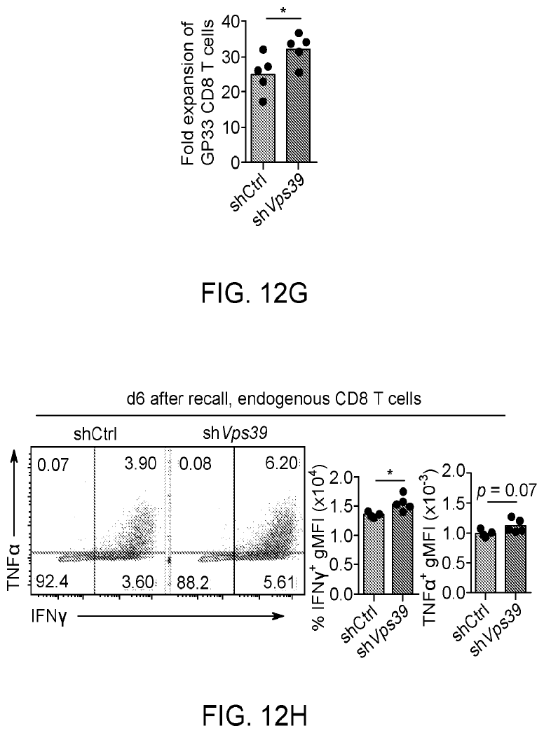

The robust expansion at primary peak responses and the reduced contraction during memory transition of Vps39-silenced SMARTA CD4 T cells prompted the further examination of whether antibody and memory CD8 T cell responses in host mice were affected. As shown in FIG. 12A, relative frequencies of CXCR5^{hi} PD-1^{hi} germinal center (GC) within adoptively transferred SMARTA CD4 T cells remained unchanged, suggesting that Vps39 silencing did not bias differentiation. However, the number of GC SMARTA TFH cells on day 8 was highly increased after Vps39 silencing (FIG. 12A). Consequently, Fas GL-7⁺ GC B cell numbers (FIG. 12B), CD138⁺IgD⁻ plasma cell numbers (FIG. 12C) and LCMV-specific serum IgG titer (FIG. 12D) were increased in mice receiving Vps39-silenced SMARTA CD4 T cells compared to mice receiving control-silenced cells. At day 30 after infection, the number of endogenous GP33 tetramer positive CD8 T memory cells increased by 2-fold in mice receiving Vps39-silenced SMARTA CD4 cells compared to mice receiving control-silenced cells (FIG. 12E and FIG. 11F). After rechallenge with Lm-GP33, these memory CD8 T cells underwent increased fold expansion and appear to have superior memory cell quality as shown by increased IFNγ production on a per-cell basis (FIG. 12F-H).

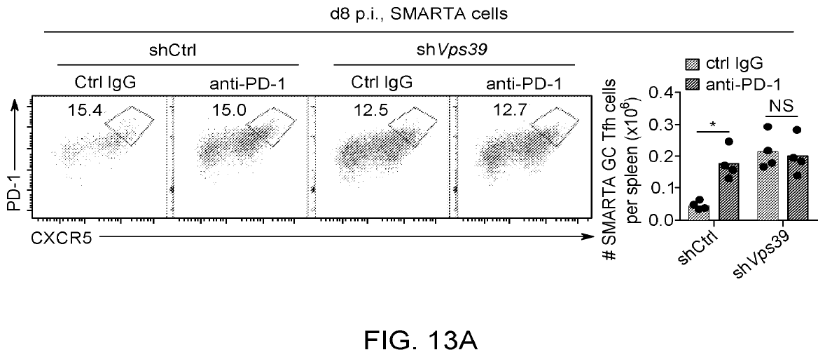

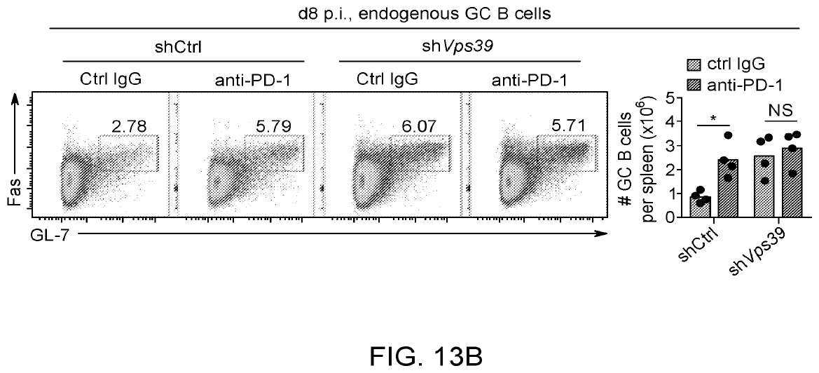

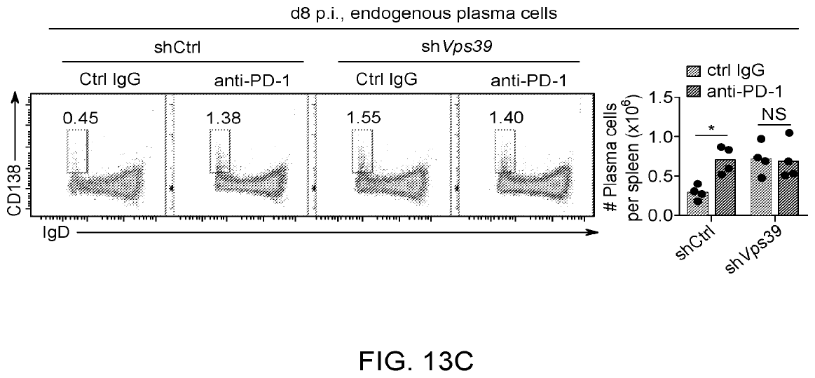

To examine whether these changes were due to reduced PD-1 expression in the adoptively transferred cells, the PD-1/PD-L1 pathway was blocked by injecting mice with anti-PD-1 blocking antibody. As shown in FIG. 13A-C, PD-1 blockade increased the numbers of transferred GC SMARTA TFH cells, endogenous GC B cells and plasma cells in mice receiving control-silenced cells while it did not further increase these numbers in mice receiving Vps39-silenced cells.

In summary, it was shown that mTORC1 signaling occurs at the late endosome and not at the lysosome in old T cells. This has important consequences for the regulation of mTORC1 activity as it changes a negative regulatory feedback to a forward loop. mTORC1 phosphorylates TFEB, thereby inhibiting transcription of lysosomal genes and impairing proteolytic activity that is required for further activation of mTORC1 at the lysosome. In contrast, reduced lysosomal activity in T cells from older adults induces the expansion of late endosomes and the expression of the leucine transporter SLC7A5, the latter by failing to degrade c-MYC. Together, increased late endosome mass and increased SLC7A5 activity support further mTORC1 activation leading to further lysosome dysfunction. Progressively dysfunctional lysosomes fail to degrade PD-1, resulting in its increased and sustained cell surface expression and inhibition of proliferation. This cycle can be broken by inhibiting or silencing SLC7A5 or VPS39 that restore TFH generation in T cells from older adults and augments the generation of germinal center and CD8 memory response in the LCMV model of infection.

Lysosomal activation of mTORC1 is controlled during cell proliferation by the cross-regulation of lysosomal and mTORC1 activities. When lysosomal activity is deficient and amino acid efflux is low, mTORC1-dependent phosphorylation of TFEB is reduced, resulting in the translocation of TFEB into the nucleus, where it stimulates lysosomal gene transcription and restores lysosomal activities; Adequate lysosomal activities trigger the mTORC1-dependent phosphorylation and cytoplasm retention of TFEB and the termination of its transcriptional induction of lysosomal genes. This feedback loop appears to be dysregulated in lysosome-deficient aged T cells as they fail to down-regulate mTORC1 to restore TFEB-dependent lysosomal genes expression; instead they show enhanced late endosomal mTORC1 activity with even more reduced lysosomal genes expression compared to the young cells.

Late endosomes are acidic organelles that, in contrast to lysosomes, do not have proteolytic activity and amino acid recycling ability. They function as temporary storage tanks for proteins that are sorted into the lysosomal degradation pathway after fusion with lysosomes or secreted as exosomes. Late endosome turnover is regulated downstream by lysosomal degradation and upstream by VPS39-mediated biogenesis from early endosomes. Inhibition of lysosomal activities induces an intracellular expansion of late endosomes. Activated T cells from older adults are deficient in rebuilding functional lysosomes as consequence of FOXO1 degradation and therefore expand this late endosomal compartment, which provides an alternative platform for mTORC1 activation. Although late endosomes do not degrade protein to recycle amino acids for mTORC1 signaling, the increased plasma membrane leucine transporter SLC7A5 induced by lysosome inhibition facilitated the uptake of extracellular amino acids as an alternative amino acid source for late endosomal mTORC1 activation. In addition to late endosomes, mTORC1 can be activated on the surface of Golgi via recruitment to the Golgi membrane, involving RAB1A and Golgi-resident RHEB. Recruitment of mTORC1 in mitochondria membrane by co-targeting Raptor and RHEB to mitochondria can also allow its activation. These lysosome-independent mTORC1 signaling may contribute to a sustained high level of mTORC1 activities as we saw in T cells from old adults in spite of impaired lysosomes, but they lack the positive feedback loop seen with late endosomal mTORC1.

One additional component in this network is activated AKT that is more sustained in naïve T cell responses of old adults due to the increased expression of miR-21 and the repression of its target phosphatase and tensin homolog (PTEN). In addition to phosphorylating tuberous sclerosis complex 2 (TSC2) and PRAS40, both negative regulators of mTOR activity, AKT also phosphorylates and inactivates FOXO1 that is needed for generation of new lysosomes. FOXO1 promotes lysosomal activities through induction of TFEB transcription in CD4 T cells. This function was impaired in T cell responses of older adults due to increased degradation of FOXO1 after T cell stimulation. Sustained mTORC1 activation at the late endosomes further inhibits lysosome activity that play an important role in promoting effector T cell survival, mainly through the autophagy/lysosome pathway.

One possible pathway how lysosome dysfunction in older adults affects T cell responses is a failure to curtail activation-induced PD-1 expression. FOXO1-deficient mouse CD4 T cells, in part recapitulating FOXO1-deficiency in activated T cells from old adults, had an up to fourfold increase of PD-1 protein levels and a major defect in T cell expansion after antigen priming in vivo. Moreover, PD-1 is degraded by lysosomes. Indeed, proliferation of T cells from old adults, which was intact after polyclonal stimulation, was impaired by increased cross-linking of PD-1 with PD-L1 compared to that of T cells from young adults.

Silencing of VPS39 to inhibit late endosome generation and activation of mTORC1 promoted T cell expansion to anti-CD3 bead stimulation in the presence of PD-L1 stimulating PD-1. A similarly enhanced expansion of VPS39-deficient T cells to stimulation of SARS-CoV-2 and pertussis peptides in the presence of antigen-specific cells was seen in vitro. The increased responses resembled those achieved by PD-1 blocking. In the acute LCMV infection model, adoptive transfer of VPS39-deficient LCMV-specific CD4 T cells resulted in increased clonal expansion, germinal center formation and improved CD8 recall responses. These results with VPS39-silenced T cells resemble results where the PD-1/PD-L1 pathway blockade enhanced both effector function and frequency of memory precursor of antigen-specific CD8 T cells and reduced viral load in acute LCMV infection. In an immunization model, PD-1 blockade promoted the generation of follicular helper T cells that are specialized in promoting cognate B cell responses. Thus, targeting VPS39 could serve as a strategy to conquer different types of virus infections and to establish the improved immune memory in older individuals. In addition to PD-1, several molecules of functional relevance are targets of lysosomal degradation. CTLA-4, another inhibitory receptor, was also reduced after enhancing lysosomal activities by late endosomal mTORC1 inhibition. However, CTLA-4 expression occurred early in a T cell response and before the observed age-related differences in mTORC1 activity.

Inhibition of late endosomal mTORC1 may serve as a strategy in compensating for age-related defects in T cell differentiation and the generation of TFH and memory cells. In addition to VPS39, there are five other protein components of HOPS complex (VPS11, VPS16, VPS18, VPS33 and VPS41) that also play a role in mediating early to late endosome conversion and could be targeted. In addition, partially inhibition of SLC7A5 to compensate for the physiological differences in expression in CD4 T cells from young and old adults could also be explored. However, complete inhibition will likely be detrimental because knockout of Slc7a5 profoundly impaired T cell activation and expansion. Based on the early studies in the mouse that mTORC1 inhibition by low dose of rapamycin produced an enhanced primary and memory recall CD8 T cell response as well as promoted follicular helper cell over Th1 cell generation, clinical studies have been started. Treatment with low dose combination of a catalytic (BEZ235) plus an allosteric (RAD001) mTOR inhibitor enhanced the immune response to the influenza vaccine and reduced the percentage of CD4 and CD8 T lymphocytes expressing PD-1 in older individuals. However, a subsequent study by resTORbio was not successful, missing the primary endpoint (www.ClinicalTrials.gov; Identifier: NCT04139915). It should be noted that the design of the study was to show an overall improvement in immune health. The vaccine response was not specifically targeted, in fact, there was a wash out period before the vaccination. Targeting mTORC1 activation on late endosomes in the days subsequent to the initiation of the T cell activation rather than mTORC1 globally may be more advantageous to stimulate T cell responses and enhance vaccine responses functions in general and in particular in older individuals.

### Example 9 Preventing Melanoma Growth Using Adoptively Transferred Vps39-Silenced CART Cells

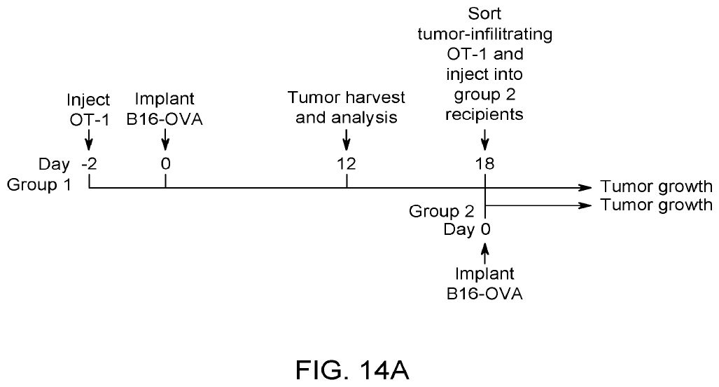

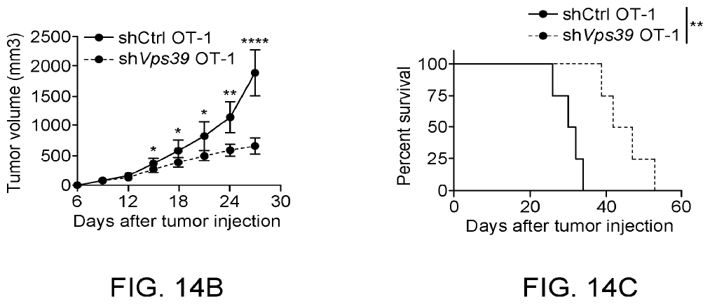

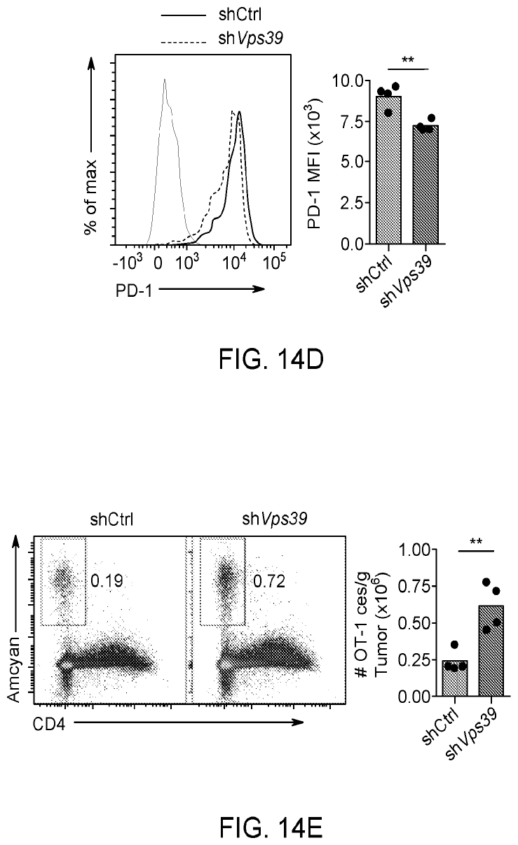

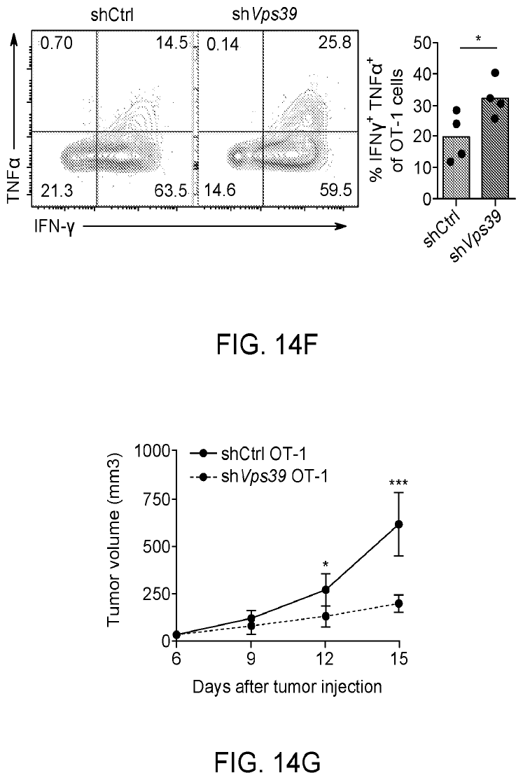

FIG. 14A provides a schematic of an experiment to prevent melanoma growth using adoptively transferred Vps39-silenced CAR-T cells. Naïve OT-I cells were activated and transduced with a retroviral vector expressing either scrambled control RNA or Vps39 shRNA. At day 6 after activation, retrovirus-transduced Amcyan⁺ OT-I cells were sorted, and 5×10⁴ transduced cells were intravenously transferred to wild-type C57BL/6 (CD45.2) mice (The Jackson Laboratory). At day 2 after transfer, mice were injected in the flank subcutaneously with 5×10⁵ B16-OVA cells (mouse melanoma cells expressing ovalbumin peptide residues 200-290, a gift from Matthew Williams at University of Utah). FIG. 14B shows the tumor growth curves for B16-OVA tumors following transfer of naive OT-1 control or Vps39-silenced CD8⁺ T cells separately into wild-type recipients. FIG. 14C shows the survival curves of the same mice. Tumor volume was significantly lower in the mice containing the adoptively transferred Vps39-silenced CD8⁺ T cells, and such mice had a significantly increased percent survival. FIG. 14D shows cell surface PD-1 expression of tumor-infiltrating Amcyan⁺ OT-I cells at day 9 after tumor implant and FIG. 14E shows the numbers of tumor-infiltrating Amcyan⁺ OT-I cells at day 9 after tumor implant. FIG. 14F shows cytokine production (TNFα and IFN-γ) of tumor-infiltrating Amcyan⁺ OT-I cells restimulated with OVA257-264 peptide ex vivo at day 9 after tumor implant. Alternatively, at day 18 after tumor implant, Amcyan⁺ tumor-infiltrating OT-I cells were sorted and injected into a new group of wild-type C57BL/6 mice followed by B16-OVA tumor implant. FIG. 14G shows tumor growth curves for B16-OVA tumors following transfer of OT-1 control or Vps39-silenced CD8⁺ T cells that had been challenged with the same tumor cell line for 18 days separately into wild-type recipients.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for treating cancer in a subject, said method comprising administering, to said subject, engineered immune cells, wherein said immune cells have reduced expression of a VPS39 polypeptide.

2. The method of claim 1, wherein said engineered immune cells are chimeric antigen receptor T cells.

3. The method of claim 1, wherein said engineered immune cells are tumor infiltrating lymphocytes.

4. The method of claim 1, wherein said cancer is melanoma.

5. A method of increasing efficacy of adoptive cell transfer in a subject, said method comprising administering to the subject chimeric antigen receptor T cells having reduced expression of a VPS39 polypeptide.

6. The method of claim 5, wherein said subject has cancer.

7. The method of claim 6, wherein said cancer is melanoma.

8. An immune cell comprising an inactivated VPS39 gene.

9. The immune cell of claim 8, wherein said immune cell is a T cell.

10. The immune cell of claim 8, wherein said T cell is a chimeric antigen receptor T cell.

11. The immune cell of claim 8, wherein said immune cell is a tumor-infiltrating lymphocyte.

12. An isolated immune cell comprising a disrupted nucleic acid encoding a VPS39 polypeptide, wherein said cell does not express an endogenous VPS39 polypeptide.

13. The immune cell of claim 12 wherein said immune cell is a T cell.

14. The immune cell of claim 12, wherein said T cell is a chimeric antigen receptor T cell.

15. The immune cell of claim 12, wherein said immune cell is a tumor-infiltrating lymphocyte.
